(19) United States
(12) Patent Application Publication
(10) Pub. No.: US 2020/0146361 A1
(43) Pub. Date: May 14, 2020

(54) VAPORIZING RELATED DATA PROTOCOLS

(71) Applicant: Pax Labs, Inc., San Francisco, CA (US)

(72) Inventors: Jesse Amos Silver, Oakland, CA (US); Amanda Adams, San Francisco, CA (US); Nicolas Paul Dor, San Francisco, CA (US); Joshua Fu, San Francisco, CA (US); Conrad Legendy, San Francisco, CA (US); Casey S. Yost, Daly City, CA (US); Aditya Palande, Saratoga, CA (US); Maya Voskoboynikov, San Francisco, CA (US); Matthew Czapar, San Francisco, CA (US); Pradyumna Upadhya, San Jose, CA (US); Ronald Buell, Piedmont, CA (US)

(21) Appl. No.: 16/679,062

(22) Filed: Nov. 8, 2019

Related U.S. Application Data
(60) Provisional application No. 62/923,901, filed on Oct. 21, 2019.
(60) Provisional application No. 62/857,615, filed on Jun. 5, 2019.
(60) Provisional application No. 62/828,244, filed on Apr. 2, 2019.
(60) Provisional application No. 62/758,432, filed on Nov. 9, 2018.

(51) Int. Cl. A24F 47/00; G06F 3/01; G06F 3/0482; H04W 4/80

(52) U.S. Cl. CPC A24F 47/008; H04W 4/80; G06F 3/0482; G06F 3/017

(57) ABSTRACT

In an aspect, there is provided an apparatus which may include a cartridge and a vaporizer body. The cartridge may include a cartridge body defining a reservoir configured to contain vaporizable material, a vaporizing assembly positioned within the cartridge body. The vaporizer body may include a receptacle configured to receive at least a portion of the cartridge, and control circuitry comprising at least one processor and at least one memory disposed within the vaporizer body, the at least one memory including instructions which when executed cause operations comprising: storing an event indicative of detection of an insertion of the cartridge into the vaporizer body; requesting, from the cartridge, cartridge information; receiving, in response to the request, the cartridge information stored at the cartridge; and configuring, based at least in part of the received cartridge information, the vaporizer device for vaporizing of the vaporizable material.

### CROSS REFERENCE TO RELATED APPLICATIONS

This application claims priority to U.S. Provisional Patent Application 62/758,432, filed Nov. 9, 2018, entitled “Vaporizing Related Data Protocols,” U.S. Provisional Patent Application 62/828,244, filed Apr. 2, 2019, entitled “Vaporizing Related Data Protocols,” U.S. Provisional Patent Application 62/857,615, filed Jun. 5, 2019, entitled “Vaporizing Related Data Protocols,” and U.S. Provisional Patent Application 62/923,901, filed Oct. 2, 2019, entitled “Vaporizing Related Data Protocols,” the contents of all of these applications are hereby incorporated by reference in their entirety.

### TECHNICAL FIELD

The current subject matter described herein relates generally to vaporizer devices, such as portable, personal vaporizer devices for generating and delivering an inhalable aerosol from one or more vaporizable materials and generally relates to data exchange protocols associated with vaporizer devices.

### BACKGROUND

Vaporizing devices, including electronic vaporizers or e-vaporizer devices, allow the delivery of vapor containing one or more active ingredients by inhalation of the vapor. Electronic vaporizer devices are gaining increasing popularity both for prescriptive medical use, in delivering medicaments, and for consumption of nicotine, tobacco, other liquid-based substances, and other plant-based smokeable materials, such as cannabis, including solid (e.g., loose-leaf) materials, solid/liquid (e.g., suspensions, liquid-coated) materials, wax extracts, and prefilled pods (cartridges, wrapped containers, etc.) of such materials. Electronic vaporizer devices in particular may be portable, self-contained, and convenient for use.

### SUMMARY

In an aspect, there is provided an apparatus. The apparatus may include a cartridge and a vaporizer body. The cartridge may include a cartridge body defining, at least in part, a reservoir configured to contain vaporizable material, a vaporizing assembly positioned within the cartridge body in fluid communication with the reservoir, the vaporizing assembly configured to vaporize the vaporizable material, and a mouthpiece coupled to a proximal end region of the cartridge body. The vaporizer body may include a receptacle configured to receive at least a portion of the cartridge, and control circuitry comprising at least one processor and at least one memory disposed within the vaporizer body, the at least one memory including instructions which when executed cause operations comprising: storing an event indicative of detection of an insertion of the cartridge into the vaporizer body; requesting, from the cartridge, cartridge information; receiving, in response to the request, the cartridge information stored at the cartridge; and configuring, based at least in part of the received cartridge information, the vaporizer device for vaporizing of the vaporizable material.

In some variations, one or more features disclosed herein including the following features can optionally be included in any feasible combination. The cartridge information may be received via a first near field communication circuitry at the cartridge and a second near field communication circuitry at the vaporizer body. The cartridge information may include a pod identifier identifying the cartridge containing the vaporizable material and/or a batch identifier indicating a composition of the vaporizable material. The operations may further include requesting, from the cartridge and after a detection of a puff of the vaporizer device, usage information of the vaporizer device and storing, at the at least one memory, the usage information received from the cartridge. The operations may further include compressing, by the vaporizer device, the cartridge information and/or the usage information after a detection of a puff, a detection of a cartridge insertion, and/or a threshold of available storage is reached. The operations may further include establishing one or more connections with a user device and/or a remote server and sending, via the one or more connections, the cartridge information and/or the usage information to the user device and/or the remote server. The usage information may include a total quantity of puffs taken from the cartridge, a total puff time for the cartridge, a total energy delivered to the cartridge, a time and date information for the puffs taken from the cartridge, and/or user feedback information regarding the vaporizable material. The receptacle may be configured to mate and electrically connect with the cartridge. The cartridge information may be stored in memory of a near field communication circuitry at the cartridge.

In another aspect, there is provided a cartridge for a vaporizer. The cartridge may include a cartridge body defining, at least in part, a reservoir configured to contain vaporizable material; a vaporizing assembly positioned within the cartridge body in fluid communication with the reservoir, the vaporizing assembly including a heating element configured to heat and cause vaporization of the vaporizable material into air drawn into a vaporizer device along an air flow path; a mouthpiece coupled to a proximal end region of the cartridge body; and circuitry comprising a controller, at least one memory storing cartridge information, and a wireless transceiver, the circuitry causing operations comprising: sending cartridge information to the vaporizer body; and sending usage information to the vaporizer body.

In some variations, one or more features disclosed herein including the following features can optionally be included in any feasible combination. The wireless transceiver may include near field communication circuitry disposed at an end of the cartridge opposite to the mouthpiece, and the at least one memory may be memory included in the near field communication circuitry. The cartridge information and/or usage information may be sent via the wireless transceiver. The cartridge information may include a pod identifier identifying a cartridge containing the vaporizable material and/or a batch identifier indicating a composition of the vaporizable material. The usage information may include a total quantity of puffs taken from the cartridge, a total puff time for the cartridge, a total energy delivered to the cartridge, a time and date information for the puffs taken from the cartridge, and/or user feedback information regarding the vaporizable material. The usage information may be sent to the vaporizer body after at least one puff of the mouthpiece and/or after a request is received from the vaporizer body. The cartridge information may be sent to the vaporizer body in response to insertion of the cartridge into the vaporizer body, after at least one puff of the mouthpiece, and/or after a request is received from the vaporizer body. The cartridge may receive the cartridge information and/or usage information. And, the cartridge information may be received from the vaporizer body, a user device coupled to the vaporizer body, and/or a remote server. Furthermore, the cartridge information and/or usage information is received via the wireless transceiver.

In another aspect, there is provided an apparatus including at least one processor and at least one memory including program code which when executed by the at least one processor causes operations comprising establishing a first connection to a vaporizer device including a cartridge, the cartridge including a vaporizable material; receiving, in response to a first request sent to the vaporizer device, a first set of cartridge information stored at the vaporizer device, the first set of cartridge information including a pod identifier uniquely identifying the cartridge, a manufacturer identifier identifying a manufacturer of the vaporizable material included in the cartridge, and strain information indicating a strain of the vaporizable material included in the cartridge; sending, in response to a second connection being established to a remote server, the first set of cartridge information to the remote server; receiving, from the remote server and in response to the sending of the first set of cartridge information to the remote server, a second set of cartridge information including batch information indicative of a composition of the vaporizable material in the cartridge; and configuring, based on the first set of cartridge information and/or the second set of cartridge information, the vaporizer device including the cartridge to vaporize the vaporizable material.

In some variations, one or more features disclosed herein including the following features can optionally be included in any feasible combination. The first connection may include a Bluetooth wireless connection. The strain information and/or the batch information may include one or more of the following: a filler identifier indicating an identity of a filler of the cartridge, a batch identifier indicating an identifying a batch of the vaporizable material; a strain identifier indicating the strain of the vaporizable material in the cartridge, a percentage of tetrahydrocannabinol contained in the vaporizable material, a percentage of cannabidiol contained in the vaporizable material, a percentage of terpenes contained in the vaporizable material, a recommended temperature for flavor when heating the vaporizable material, and/or a recommended temperature for vapor when heating the vaporizable material. The first set of cartridge information may include a strain identifier and/or a batch identifier. The first set of cartridge information may also include one or more of the following: usage information, and wherein the usage information includes a total quantity of puffs taken from the cartridge, a total puff time for the cartridge, a total energy delivered to the cartridge, a time and date information for the puffs taken from the cartridge, and/or user feedback information regarding the vaporizable material. The first request may indicate to the vaporizer device to provide a certain type of cartridge information, provide updated cartridge information, or provide all cartridge information available at the vaporizer device including the cartridge. The sending may be in response to a second request received from the remote server. The second request may indicate to the user device including the mobile application to provide to the remote server a certain type of cartridge information, updated cartridge information, or all cartridge information available at the user device including the mobile application. The operations may further include receiving, from the remote server and in response to the sending of the first set of cartridge information to the remote server, a lock indication to lock operation of the user device and/or the vaporizer device to prevent vaporization of the vaporizable material. The apparatus may include a smart phone, a tablet, and/or a computer. The cartridge receptacle may be configured to mate and electrically connect with the cartridge. At least a portion of the first set of cartridge information and/or a portion of the second cartridge information may include a user preference information provided by a user to the apparatus and/or a remote server. The user preference information may include a recommended target temperature for flavor and/or a recommended temperature for vapor.

In another aspect, there is provided a system include at least one processor; at least one memory including program code, which when executed by the at least one processor causes operations comprising: receiving, from a first user device, information indicating a strain of a vaporizable material included in a cartridge configured to contain the vaporizable material; sending, to the first user device, a pod identifier to enable a filler of the cartridge to associate the pod identifier to the cartridge; receiving, from the first user device and after the cartridge is filled with the vaporizable material, a strain identifier mapped to the pod identifier and/or to a batch identifier, the strain identifier indicating the strain of the vaporizable material included in the filled cartridge; and storing the pod identifier mapped to the strain identifier to enable configuration of a vaporizer device.

In some variations, one or more features disclosed herein including the following features can optionally be included in any feasible combination. The batch identifier includes information indicating a composition of the vaporizable material included in the cartridge. The batch information may include a filler identifier indicating an identity of the filler of the cartridge, the strain identifier indicating the strain of the vaporizable material in the cartridge, a percentage of tetrahydrocannabinol contained in the vaporizable material, a percentage of cannabidiol contained in the vaporizable material, a percentage of terpenes contained in the vaporizable material, a recommended temperature for flavor when heating the vaporizable material, and a recommended temperature for vapor when heating the vaporizable material. The first user device may be coupled to a filling machine configured to fill the cartridge with the vaporizable material, write the pod identifier in a memory at the cartridge, and write the strain identifier in the memory at the cartridge. The first user device may be coupled to a near field communication writer to write and/or read to the memory at the cartridge the batch information. The operations may further include receiving usage information associated with the vaporizer device, wherein the usage information includes a total quantity of puffs taken from the cartridge, a total puff time for the cartridge, a total energy delivered to the cartridge, a time and date information for the puffs taken from the cartridge, and/or user feedback information regarding the vaporizable material. The operations may further include providing, to a second user device coupled to the vaporizer device, the usage information, the pod identifier, and/or the strain identifier to enable the second user device to configure the vaporizer device for vaporization of the vaporizable material in the cartridge. The operations may further include sending, to the second user device coupled to the vaporizer device, a lock indication to lock operation of the second user device and/or the vaporizer device to prevent vaporization of the vaporizable material. Details of one or more variations of the subject matter described herein are set forth in the accompanying drawings and the description below. Other features and advantages of the subject matter described herein will be apparent from the description and drawings, and from the claims.

In another aspect, there is provided an apparatus. The apparatus may include display and at least one processor and at least one memory including program code which when executed by the at least one processor causes the apparatus to at least: establish a first connection to a vaporizer device including a cartridge, the cartridge including a vaporizable material; send, to a remote server, a request for test information for the vaporizable material; receive, from the remote server and in response to the request, test information indicating a provenance of the vaporizable material, when the remote server includes the provenance information for the vaporizable material; and present, at the display, a user interface view including the test information.

In some variations, one or more features disclosed herein including the following features can optionally be included in any feasible combination. The apparatus may be further caused to at least display a warning, when the remote server does not include the provenance information for the vaporizable material. The apparatus may be further caused to at least inhibit vaporization of the vaporizer material, when the remote server does not include the provenance information for the vaporizable material. The request may include, for the vaporizable material, a pod identifier, a batch identifier, and/or a strain identifier.

In another aspect, there is provided a server. The server may include at least one processor and at least one memory including program code, which when executed by the at least one processor causes the server to at least: receive, for a vaporizable material, test information for a batch of a strain of the vaporizable material; receive, from a user device, a request for test information for the vaporizable material, the user device coupled to a vaporizer device including a cartridge, the cartridge including a vaporizable material; search for test information for the vaporizable material; generate a response including the test information, when search results include the test information for the vaporizable material; and send, to the user device, the generated response including the test information to enable the user device to authenticate a provenance of the vaporizable material.

In some variations, one or more features disclosed herein including the following features can optionally be included in any feasible combination. The server may be further caused to at least send, to the user device, an indication that test information for the vaporizable material is not available at the server to enable the user device to detect a lack of the provenance of the vaporizable material. The indication may be a warning for presentation at the user device and/or for inhibition of vaporization of the vaporizable material. The test information may include a certificate of analysis for the vaporizable material. The request may include, for the vaporizable material, a pod identifier, a batch identifier, and/or a strain identifier.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings:

FIG. 1A-FIG. 1F illustrate features of a vaporizer device including a vaporizer body and a cartridge consistent with implementations of the current subject matter;

FIG. 2 is a block diagram illustrating features of a vaporizer device having a cartridge and a vaporizer body consistent with implementations of the current subject matter;

FIG. 3 illustrates communication between a vaporizer device, a user device, and a server consistent with implementations of the current subject matter;

FIG. 4 depicts an example of a data exchange sequence associated with a vaporizer consistent with implementations of the current subject matter;

FIG. 5 depicts an example of a data exchange sequence including compression consistent with implementations of the current subject matter;

FIG. 6 depicts an example of a data exchange sequence between the vaporizer, a user device, and a remote server consistent with implementations of the current subject matter;

FIG. 7 depicts an example of a data exchange sequence between a cartridge manufacturer and a remote server consistent with implementations of the current subject matter;

FIG. 8 depicts an example of a data exchange sequence between a cartridge filler and a remote server consistent with implementations of the current subject matter;

FIG. 9A depicts an example of a system handling data exchanges related to aspects of the vaporizer;

FIG. 9B depicts a dataflow for the system of FIG. 9A;

FIG. 10A depicts an example of a user interface presented at partner portal;

FIG. 10B depicts another example of a user interface presented at the partner portal;

FIG. 11A depicts a user interface presented at a production portal;

FIG. 11B depicts another user interface presented on the production portal;

FIG. 12 depicts an example of a user interface presented on user device;

FIG. 13A-B depicts examples of a user interface providing analytics;

FIG. 14 depicts an example of a process for enabling fillers to obtain usage data regarding cartridges consistent with implementations of the current subject matter;

FIG. 15 depicts an example of a process for receiving cartridge information consistent with implementations of the current subject matter;

FIG. 16 depicts an example of a process for providing stored cartridge information to a vaporizer consistent with implementations of the current subject matter;

FIG. 17 depicts an example of a process at a user device such as a smartphone or other processor consistent with implementations of the current subject matter;

FIG. 18 depicts an example of a process at a user device such as a smartphone or other type of processor consistent with implementations of the current subject matter;

FIG. 19 depicts an example of a process configured at a user device for handling certificate of analysis information representative of testing information for a vaporizable material in a cartridge inserted into a vaporizable body consistent with implementations of the current subject matter;

FIGS. 20A-20C depict examples of user interface views including strain information and/or batch information for a vaporizable material contained in a cartridge consistent with implementations of the current subject matter;

FIG. 21 depicts an example of a user interface view including a portion of the certificate of analysis information presented as test information consistent with implementations of the current subject matter; and

FIG. 22 depicts an example of a process configured at a remote server for providing certificate of analysis information representative of testing information for a vaporizable material in a cartridge inserted into a vaporizable body consistent with implementations of the current subject matter.

When practical, similar reference numbers denote similar structures, features, or elements.

### DETAILED DESCRIPTION

Implementations of the current subject matter include devices relating to vaporizing of one or more materials for inhalation by a user. The term “vaporizer” may be used generically in the following description and may refer to a vaporizer device, such as an electronic vaporizer. Examples of vaporizers consistent with implementations of the current subject matter include electronic vaporizers, electronic cigarettes, e-cigarettes, or the like. In general, such vaporizers are often portable, hand-held devices that heat a vaporizable material to provide an inhalable dose of the material. Vaporizers consistent with the current subject matter may be referred to by various terms such as inhalable aerosol devices, aerosolizers, vaporization devices, electronic vaping devices, electronic vaporizers, vape pens, etc. For example, the vaporizer may include a heater configured to heat a vaporizable material that results in the production of one or more gas-phase components of the vaporizable material. A vaporizable material may include liquid and/or oil-type plant materials. The gas-phase components of the vaporizable material may condense after being vaporized such that an aerosol is formed in a flowing air stream that is deliverable for inhalation by a user. The vaporizer devices may, in some implementations of the current subject matter, be particularly adapted for use with an oil-based vaporizable material, such as cannabis-derived oils although other types of vaporizable materials may be used as well.

One or more features of the current subject matter, including one or more of a cartridge (also referred to as a vaporizer cartridge or pod) and a reusable vaporizer device body (also referred to as a vaporizer device base, a body, a vaporizer body, or a base), may be employed with a suitable vaporizable material (where suitable refers in this context to being usable with a device whose properties, settings, etc. are configured or configurable to be compatible for use with the vaporizable material). The vaporizable material may include one or more liquids, such as oils, extracts, aqueous or other solutions, etc., of one or more substances that may be desirably provided in the form of an inhalable aerosol.

The cartridge may be inserted into a vaporizer's body, and then the vaporizable material heated which results in the inhalable aerosol. The vaporizable material may comprise a variety of ingredients each of which may have, for example, a different target temperature to attain vaporization. Moreover, a user of the vaporizer may have different preferences with respect to target temperature and other aspects of the vaporizer's configuration to achieve a desired vaping experience. Furthermore, a filler of the cartridge or a maker of the vaporizer material may have a certain recommended vaporizer configuration for each vaporizable material. In some instances, the filler and the maker are the same entity, while in some instances the maker and filler are separate entities. Likewise, the manufacturer or supplier of the vaporizer may also have recommended vaporizer configurations. In addition, vaporizer users may share suggested vaporizer configurations, such as target temperature for a given vaporizable material. As such, the configuration of a vaporizer may require a substantial amount of information. To that end, there is provided a data exchange protocol to facilitate communication among a cartridge, a vaporizer device body, a user device (e.g., a smart phone, tablet, computer, etc. coupled to the vaporizer), a remote server, a filler of cartridges, a filler of the cartridges, and/or a maker of vaporizers or cartridges. This data exchange protocol may, in certain implementations, efficiently exchange information, which may conserve power consumption especially in a battery-operated vaporizer.

Moreover, the data exchange disclosed herein may provide a more customized vaping experience. In the case of cannabis-based vaporizable materials, the vaping session may be configured specifically for a given user. To illustrate further, the target temperature at which the vaporizable material is heated, the rate at which the temperature is raised to the target temperature, the duration of a vaping session, and/or other aspects of the vaping session may be configured using the data exchange protocol. And, this configuration may take into the account data provided by a cartridge filler, maker of the vaporizer material, vaporizer device manufacture, and other users to provide a specific user experience and/or to take into account the strain of vaporizable material.

Before providing additional details regarding the data exchange protocol, the following provides a description of some example of vaporizer devices including a vaporizer body and a cartridge.

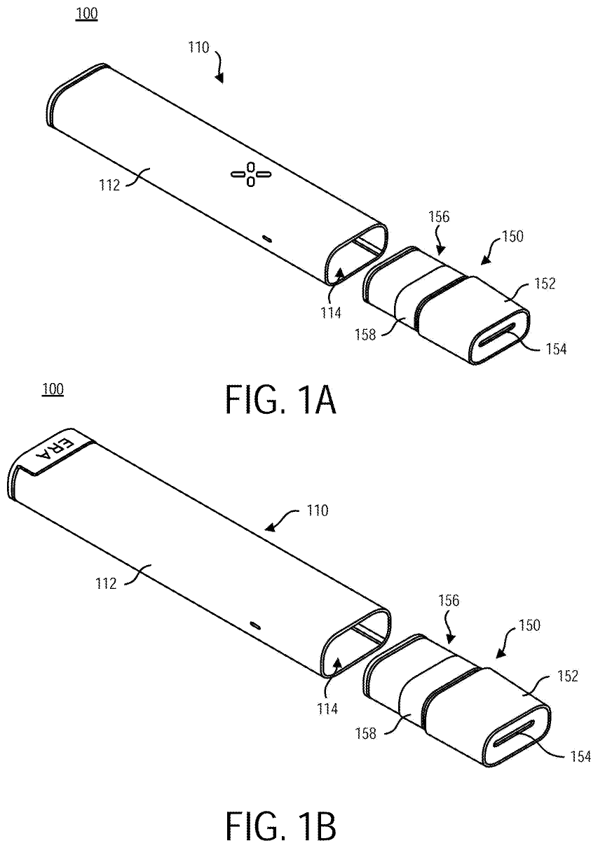

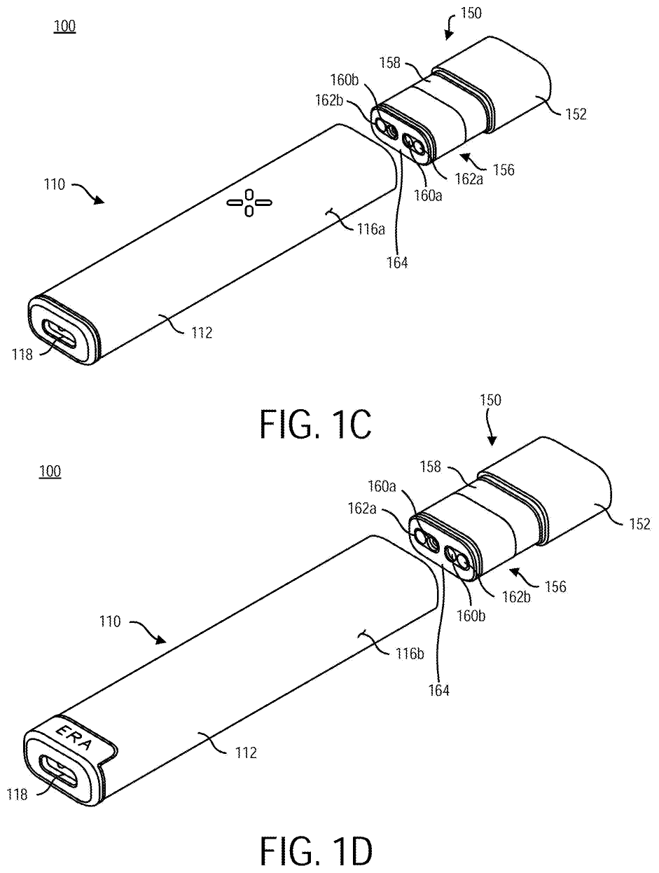

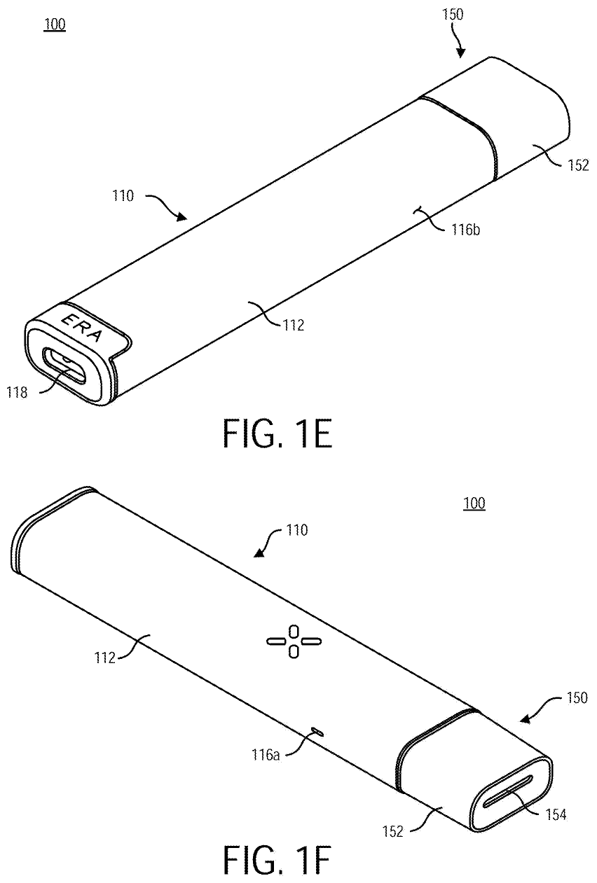

FIG. 1A-FIG. 1F illustrate features of a vaporizer device 100 including a vaporizer body 110 and a cartridge 150 consistent with implementations of the current subject matter. FIG. 1A is a bottom perspective view, and FIG. 1B is a top perspective view of the vaporizer device 100 with the cartridge 150 separated from a cartridge receptacle 114 on the vaporizer body 110. Both of the views in FIG. 1A and FIG. 1B are shown looking towards a mouthpiece 152 of the cartridge 150. FIG. 1C is a bottom perspective view, and FIG. 1D is a top perspective view of the vaporizer device with the cartridge 150 separated from the cartridge receptacle 114 of the vaporizer body 110. FIG. 1C and FIG. 1D are shown looking toward the distal end of the vaporizer body 110. FIG. 1E is a top perspective view, and FIG. 1F is a bottom perspective view of the vaporizer device 100 with the cartridge 150 engaged for use with vaporizer body 110.

As shown in FIG. 1A-FIG. 1D, the cartridge 150 includes, at the proximal end, a mouthpiece 152 that is attached over a cartridge body 156 that forms a reservoir or tank 158 that holds a vaporizable material. The cartridge body 156 may be transparent, translucent, opaque, or a combination thereof. The mouthpiece 152 may include one or more openings 154 (see FIG. 1A, FIG. 1B, FIG. 1F) at the proximal end out of which vapor may be inhaled, by drawing breath through the vaporizer device 100. The distal end of the cartridge body 156 may couple to and be secured to the vaporizer body 110 within the cartridge receptacle 114 of the vaporizer body 110. Power pin receptacles 160a,b (see FIG. 1C, FIG. 1D) of the cartridge 150 mate with respective power pins or contacts 122a,b of the vaporizer body 110 that extend into the cartridge receptacle 114. The cartridge 150 also includes air flow inlets 162a,b on the distal end of the cartridge body 156.

A data tag, such as a near-field communication (NFC) tag or other type of wireless transceiver or communication tag, 164 may be positioned on at least a portion of the distal end of the cartridge body 156, although it may be positioned in other locations as well. As shown in FIG. 1C and FIG. 1D, the NFC tag 164 may substantially surround the power pin receptacles 160a,b and the air flow openings 162a,b, although other configurations of the NFC tag may be implemented as well. For example, the NFC tag 164 may be positioned between the power pin receptacle 160a and the power pin receptacle 160b, or the NFC tag 164 may be shaped as a circle, partial circle, oval, partial oval, or any polygonal shape encircling or partially encircling the power pin receptacles 160a,b and the air flow openings 162a,b or a portion thereof.

In the example of FIG. 1A, the vaporizer body 110 has an outer shell or cover 112 that may be made of various types of materials, including for example aluminum (e.g., AL6063), stainless steel, glass, ceramic, titanium, plastic (e.g., Acrylonitrile Butadiene Styrene (ABS), Nylon, Polycarbonate (PC), Polyethersulfone (PESU), and the like), and any hard, durable material. The proximal end of the vaporizer body 110 includes an opening forming the cartridge receptacle 114, and the distal end of the vaporizer body 110 includes a connection 118, such as a universal serial bus Type C (USB-C) connection and/or the like. The cartridge receptacle 114 portion of the vaporizer body 110 includes one or more openings (air inlets) 116a,b that extend through the outer shell 112 to allow air flow therein, as described in more detail below. The vaporizer body 110 as shown has an elongated, flattened tubular shape that is curvature-continuous, although the vaporizer body 110 is not limited to such a shape. The vaporizer body 110 may take the form of other shapes, such as a rectangular box, a cylinder, and the like.

The cartridge 150 may fit within the cartridge receptacle 114 by a friction fit, snap fit, and/or other types of secure connection. The cartridge 150 may have a rim, ridge, protrusion, and/or the like for engaging a complimentary portion of the vaporizer body 110. While fitted within the cartridge receptacle 114, the cartridge 150 may be held securely within but still allow for being easily withdrawn to remove the cartridge 150.

Although FIG. 1A-FIG. 1F illustrate a certain configuration the vaporizer device 100, the vaporizer may take other configurations as well.

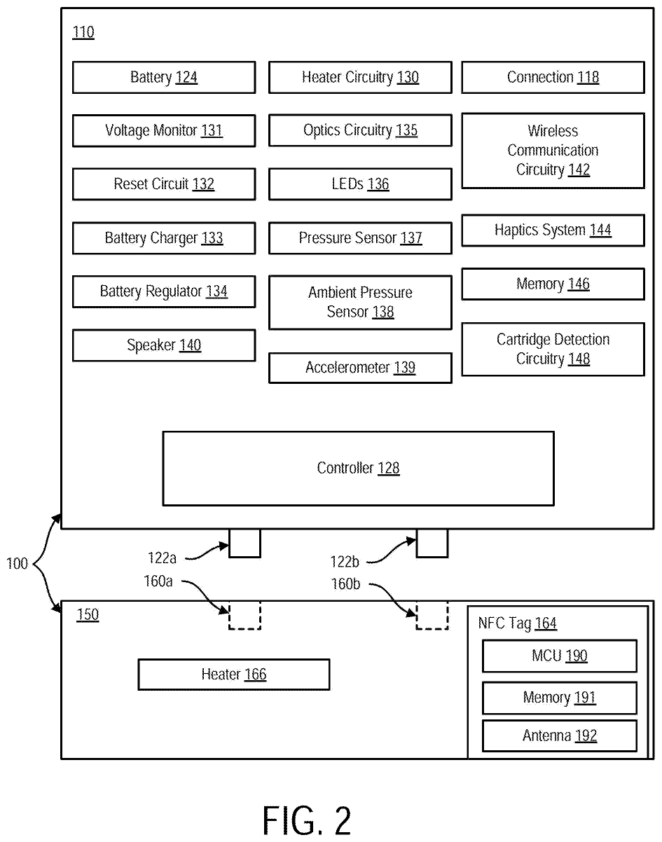

FIG. 2 is a schematic block diagram illustrating components of a vaporizer device 100 having a cartridge 150 and a vaporizer body 110 consistent with implementations of the current subject matter. Included in the vaporizer body 110 is a controller 128 that includes at least one processor and/or at least one memory configured to control and manage various operations among the components of the vaporizer device 100 described herein.

Heater circuitry 130 of the vaporizer body 110 controls a heater 166 of the cartridge 150. The heater 166 may generate heat to provide vaporization of the vaporizable material. For example, the heater 166 (which may be contained within the pod 150) may include a heating coil (e.g., a resistive heater) in thermal contact with a wick, which includes, or absorbs, the vaporizable material.

A battery 124 is included in the vaporizer body 110, and the controller 128 may control and/or communicate with a voltage monitor 131 circuitry configured to monitor the battery voltage, a reset circuit 132 configured to reset (e.g., shut down the device 100 and/or restart the device 100 in a certain state), a battery charger 133, and a battery regulator 134 (which may regulate the battery output, regulate charging/discharging of the battery, and provide alerts to indicate when the battery charge is low, etc.).

Power pins 122a,b of the vaporizer body 110 engage complementary power pin receptacles 160a,b of the cartridge 150 when the cartridge 150 is engaged with the vaporizer body 110. Alternatively, the power pins may be part of the cartridge 150 for engaging complementary power pin receptacles of the vaporizer body 110. The engagement allows for the transfer of energy from an internal power source (e.g., the battery 124) to the heater 166 in the cartridge 150. The controller 128 may regulate the power flow (e.g., an amount or current and/or a voltage amount) to control a temperature at which the heater 166 heats a vaporizable material contained in the reservoir 158. According to implementations of the current subject matter, a variety of electrical connectors other than a pogo-pin and complementary pin receptacle configuration may be used to electrically connect the vaporizer body 110 and the cartridge 150, such as for example, a plug and socket connector.

The controller 128 may control and/or communicate with optics circuitry 135 (which controls and/or communicates with one or more displays such as LEDs 136 that can provide user interface output indications), a pressure sensor 137, an ambient pressure sensor 138, an accelerometer 139, and/or a speaker 140 configured to generate sound or other feedback to a user.

The pressure sensor 137 may be configured to sense a user drawing (e.g., taking a puff, inhaling, etc.) on the mouthpiece 152 and activate the heater control circuitry 130 of the vaporizer body 110 to accordingly control the heater 166 of the cartridge 150. In this way, the amount of current supplied to the heater 166 may be varied according the user's draw (e.g., additional current may be supplied during a draw, but reduced when there is not a pull or drag taking place). The ambient pressure sensor 138 may be included for atmospheric reference to reduce sensitivity to ambient pressure changes and may be utilized to reduce false positives potentially detected by the pressure sensor 137 when measuring draws from the mouthpiece 154.

The accelerometer 139 (and/or other motion sensors, capacitive sensors, flow sensors, strain gauge(s), or the like) may be used to detect user handling and interaction, for example, to detect movement of the vaporizer body 110 (such as tapping, rolling, and/or any other deliberate movement associated with the vaporizer body 110). The detected movements may be interpreted by the controller 128 as one or more predefined user commands. For example, one particular movement may be a user command to gradually increase the temperature of the heater 166 as the user intends to begin using the device 100.

The vaporizer body 110, as shown in FIG. 2, includes wireless communication circuitry 142 that is connected to and/or controlled by the controller 128. The wireless communication circuitry 142 may include a near-field communication (NFC) antenna that is configured to read from and/or write to the NFC tag 164 of the cartridge 150. Alternatively or additionally, the wireless communication circuitry 142 may be configured to automatically detect a cartridge 150 as it is being inserted into the vaporizer body 110. In some implementations, data exchanges between the vaporizer body 110 and pod 150 take place over NFC.

The wireless communication circuitry 142 may include additional components including circuitry for other communication technology modes, such as Bluetooth circuitry, Bluetooth Low Energy circuitry, Wi-Fi circuitry, cellular (e.g., LTE, 4G, and/or 5G) circuitry, and associated circuitry (e.g., control circuitry), for communication with other devices. For example, the vaporizer body 110 may be configured to wirelessly communicate with a remote processor (e.g., a smartphone, a tablet, a computer, wearable electronics, a cloud server, and/or processor based devices) through the wireless communication circuitry 142, and the vaporizer body 110 may, through this communication, receive information including control information (e.g., for setting temperature, resetting a dose counter, etc.) from and/or transmit output information (e.g., dose information, operational information, error information, temperature setting information, charge/battery information, etc.) to one or more of the remote processors.

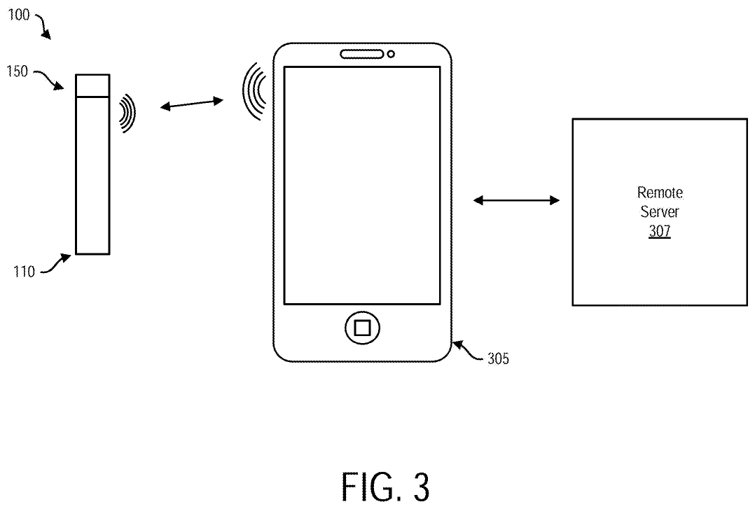

The NFC tag 164 may be a type of wireless transceiver and may include a processor such as a microcontroller unit (MCU) 190, a memory 191, and an antenna 192 (e.g., an NFC antenna) to perform the various functionalities described below with further reference to FIG. 3. NFC tag 164 may be, for example, a 1 Kbit or a 2 Kbit tag that is of type ISO/IEC 15693. NFC tags with other specifications may also be used. The NFC tag 164 may be implemented as active NFC enabling reading and/or writing information via NFC with other NFC compatible devices including a remote processor, another vaporizer, and/or wireless communication circuitry 142. Alternatively, the NFC tag 164 may be implemented using passive NFC technology, in which case other NFC compatible devices (e.g., a remote processor, another vaporizer, and/or wireless communication circuitry 142) may only be able to read information from the NFC tag 164.

The vaporizer body may include a haptics system 144, such as an actuator, a linear resonant actuator (LRA), an eccentric rotating mass (ERM) motor, or the like that provide haptic feedback such as a vibration as a “find my device” feature or as a control or other type of user feedback signal. For example, using an app running on a user device (such as the user device 305), a user may indicate that he/she cannot locate his/her vaporizer device 100. Through communication via the wireless communication circuitry 142, the controller 128 sends a signal to the haptics system 144, instructing the haptics system 144 to provide haptic feedback (e.g., a vibration). The controller 128 may additionally or alternatively provide a signal to the speaker 140 to emit a sound or series of sounds. The haptics system 144 and/or speaker 140 may also provide control and usage feedback to the user of the vaporizer device 100; for example, providing haptic and/or audio feedback when a particular amount of a vaporizable material has been used or when a period of time since last use has elapsed. Alternatively or additionally, haptic and/or audio feedback may be provided as a user cycles through various settings of the vaporizer device 100. Alternatively or additionally, the haptics system 144 and/or speaker 140 may signal when a certain amount of battery power is left (e.g., a low battery warning and recharge needed warning) and/or when a certain amount of vaporizable material remains (e.g., a low vaporizable material warning and/or time to replace the cartridge). Alternatively or additionally, the haptics system 144 and/or speaker 140 may also provide usage feedback and/or control of the configuration of the vaporizer (e.g., allowing the change of a configuration, such as target heating rate, heating rate, etc.

The vaporizer body 110 may include circuitry for sensing/detecting when a cartridge 150 is connected and/or removed from the vaporizer body 110. For example, cartridge-detection circuitry 148 may determine when a cartridge 150 is connected to the vaporizer body 110 based on an electrical state of the power pins 122a,b within the cartridge receptacle 114. For example, when the cartridge is present, there may be a certain voltage, current, and/or resistance associated with the power pins 122a,b, when compared to when the cartridge is not present. Alternatively or additionally, the NFC tag 164 may also be used to detect when the cartridge 150 is connected to the vaporizer body 110.

The vaporizer body 110 may also include the connection (e.g., USB-C connection, micro-USB connection, and/or other types of connectors) 118 for coupling the vaporizer body to a charger to enable charging the internal battery 124. Alternatively or additionally, electrical inductive charging (also referred to as wireless charging) may be used, in which case the device 110 would include inductive charging circuitry to enable charging. The connection 118 at FIG. 2 may also be used for a data connection between a computing device and the controller 128, which may facilitate development activities such as programming and debugging, for example.

The vaporizer body 110 may also include a memory 146 that is part of the controller 128 or is in communication with the controller 128. The memory 146 may include volatile and/or non-volatile memory or provide data storage. In some implementations, the memory 146 may include 8 Mbit of flash memory, although the memory is not limited to this and other types of memory may be implemented as well.

FIG. 3 illustrates communication between a vaporizer device 100 (including the vaporizer body 110 and the cartridge 150), a user device 305 (e.g., a smartphone, a tablet, a laptop, a smartwatch, a computer, and/or the like), and a remote server 307 (e.g., a server coupled to a network, a cloud server coupled to the Internet, and/or the like) consistent with implementations of the current subject matter. The user device 305 wirelessly communicates with the vaporizer device 100. The remote server 307 may communicate directly with the vaporizer device 100 or through the user device 305. The vaporizer body 110 may communicate with other devices via the wireless communication circuitry 142. In some implementations, the cartridge 150 may establish through the NFC tag 164 communication with the vaporizer body, the user device 305, and/or the remote server 307. Although FIG. 3 depicts the vaporizer 100 connecting through the remote server 307 via the user device 305, the vaporizer may be configured to establish one or more connections to the remote server directly without the user device.

An application software (“app”) running on at least one of the remote processors (the user device 305 and/or the remote server 307) may be configured to control operational aspects of the vaporizer device 100 and receive information relating to operation of the vaporizer device 100. For example, the app may provide a user with capabilities to input or set desired properties or effects, such as a particular temperature or desired dose, which is then communicated to the controller 138 of the vaporizer body 110 through the wireless communication circuitry 142. The app may also provide a user with functionality to select one or more sets of suggested properties or effects that may be based on the particular type of vaporizable material in the cartridge 150. For example, the app may allow adjusting heating based on the type of vaporizable material, the user's (of device 100) preferences or desired experience, and/or the like.

In some implementations, the app may provide to the remote server 307 and/or the user device 305 information related to the vaporizer. The provided data may include information identifying the pod 150 (e.g., pod ID), information identifying the composition of the vaporizable material in the pod (e.g., strain ID, batch ID, and/or product ID), information identifying the vendor of the pod (e.g., vendor ID), usage information, such as puffs taken (puff count), time of day of the puff, an amount of energy (e.g., in joules) applied to the vaporizable material, an amount of the vaporizable material in the pod (e.g., weight, etc.) currently or when initially filled, user experience information (e.g., user's perceived experience caused by a puff, such as calm, alert, like, dislike, etc.), and/or pod configuration (e.g., a target heater temperature to achieve vaporization, a ramp rate to the target temperate, etc.).

The app of the user device 305 may allow a user to perform a hard-reset of the vaporizer device 100. For example, a user may indicate through the app that the vaporizer device should be reset, which may cause the vaporizer device 100 to shut down, which may be performed by the reset circuit 132. Following shut-down, the vaporizer device 100 may enter a standby mode or may resume operation, depending upon a variety of factors, such as for example the reason (if known) for the reset. The input and/or user selections may act as control signals for the controller 128 to perform a corresponding function (e.g., reach and hold a defined temperature, provide a certain dose, reduce heat after a certain time period, reset, etc.). Likewise, the controller 128 may transmit information, through the wireless communication circuitry 142, to one of the remote processors for display via the app. For example, a summary of use of the vaporizer device 100 throughout a day may be tracked and sent to the user device 305.

Data read from the NFC tag 164 from the wireless communication circuitry 142 of the vaporizer body 110 may be transferred to one or more of the remote processors (e.g., the user device 305 and/or the remote server 307) to which it is connected, which allows for the app running on the user device to access and utilize the read data for a variety of purposes. For example, the read data relating to the cartridge 150 may be used for providing recommended temperatures, dose control, usage tracking, and/or assembly information.

The cartridge 150 may also communicate directly, through the NFC tag 164, with other devices. This enables data relating to the cartridge to be written to/read from the NFC tag 164, without interfacing with the vaporizer body 110. The NFC tag 164 thus allows for identifying information (e.g., pod ID, batch ID, etc.) related to the cartridge 150 to be associated with the cartridge 150 by one or more remote processors. For example, when the cartridge 150 is filled with a certain type of vaporizable material, this information may be transmitted to the NFC tag 164 and written to memory 191 by filling equipment. Then, the vaporizer body 110 is able to obtain this information from the NFC tag 164 (e.g., via circuitry 142 at the vaporizer body 110) to identify the vaporizable material currently being used and accordingly adjust the controller 128 based on, for example, user-defined criteria or pre-set parameters associated with the particular type of vaporizable material (set by a manufacturer or as determined based upon user experiences/feedback aggregated from other users). For example, a user may establish (via the app at the user device 305) a set of criteria relating to desired effects for or usage of one or more types of vaporizable materials. When a certain vaporizable material is identified, based on communication via the NFC tag 164, the controller 128 accordingly adopts the established set of criteria, which may include, for example, temperature and dose, for that particular vaporizable material.

Other information related to the cartridge 150 may be transmitted to and stored on the NFC tag 164, such as information relating to components of the cartridge 150, for example heating components. The controller 128 of the vaporizer body 110 may use this information to control a usage session for a user. A manufacturer or filler of the pod/cartridge may thus transmit manufacturing information to the NFC tag 164 for storage for subsequent use by the controller 128 or other remote processors (e.g., the user device 305 and/or the remote server 307).

In some implementations, a variety of data associated with the pod/cartridge 150 may be stored on the NFC tag 164. Examples of the types of stored data include manufacturing data (e.g., tag serial number, tag manufacturer identifier, tag IC product code, cartridge serial number, cartridge hardware revision code, date of assembly, manufacture (MFG) lot code, MFG test equipment serial number (S/N), MFG test data (e.g., coil resistance, leak/flow rate test, cosmetic check, etc.), MFG test parameters, material logging (e.g., coil type, wick type, etc.), and/or mass of empty cartridge); filler data (which may be added after the cartridge is filled with a vaporizable material, for example, batch identifier (ID), vendor ID, product ID, strain code (e.g., an identifier indicating the strain of the cannabis), mass of filled cartridge, viscosity, default/min/max temperature setting, tetrahydrocannabinol (THC) content percentage (%), cannabidiol (CBD) %, terpene %, extraction method, and/or fill date); and/or usage data (e.g., total puffs taken, total puff time, drop count, total energy delivered to cartridge (joules), date of first/most recent puff, cartridge lock (for locking cartridge to specific device/child lock), cartridge kill (initiating lock out of cartridge), min/max temperature set by user/device, min/max “baseline” resistance measured, count of bad connections (where cartridge did not properly dock and measure baseline resistance), and/or various device error codes). The tag data may be encrypted and/or hashed, and the NFC tag 164 may be password protected. In some implementations, the following may be written to the NFC tag's memory in the cartridge: vendor ID, product ID (or Strain ID), a default temperature for the heater, a recommended temperature for flavor, a recommend temperature for vapor, a pod type, vaporizer session information (e.g., a session ID, session size, lockout time), composition of vaporizable material (e.g., terpene percentage, THC percentage, CBD percentage, and test date for that composition), and/or viscosity of the vaporizable material.

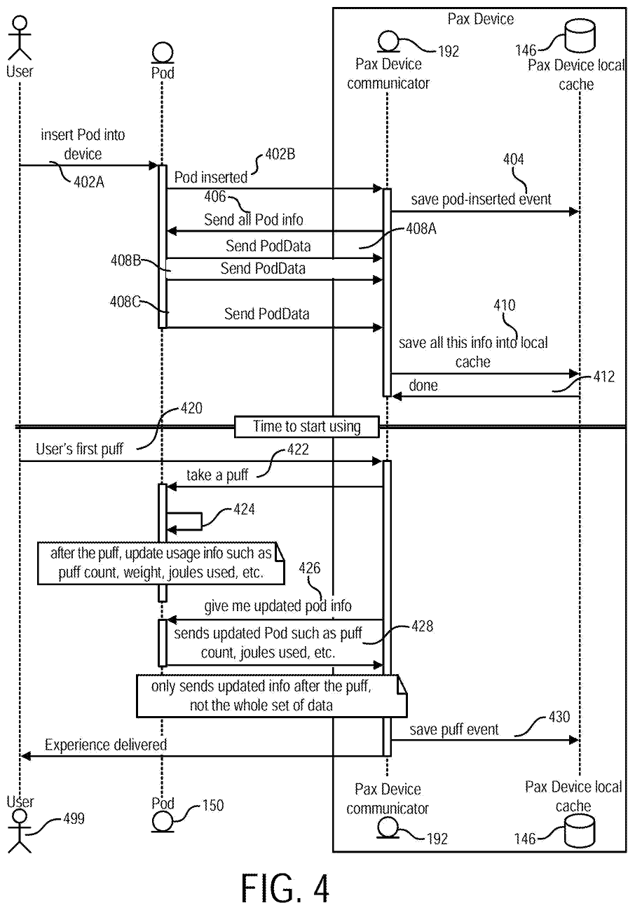

FIG. 4 depicts an example of a data exchange sequence 400 associated with vaporizer 100.

At 402A, the pod 150 is inserted into the vaporizer body 110, and this insertion may be detected, at 402B, by the vaporizer body 110. When the pod 150 is inserted into the vaporizer body 110, the pod power pins 122a,b (see, e.g., FIG. 2) of the vaporizer body 110 engage complementary power pin receptacles 160a,b of the cartridge 150. This engagement may be detected by circuitry at the vaporizer body. The insertion of the pod into the vaporizer body may be detected in other ways as well, such as via NFC or other electrical or mechanical mechanisms.

At 404, the vaporizer body 110 generates an event (e.g., save pod-inserted event at a given time) for the insertion of the pod 150 into the vaporizer device 110 and saves the insertion event information to memory 146 (labeled “local cache”). In the example of FIG. 4, the memory is a local, persistent memory 146, although other types of memory may be used as well. The insertion event information may include the pod ID (e.g., an identifier that uniquely identifies pod 150 from other pods). In some implementations, each pod 150 has a unique identifier loaded into memory 191 of the NFC tag 162 (FIG. 2). For example, a maker of the vaporizer material or a filler of the pod 150 may write a unique pod ID into memory 191 to enable tracking of the pod 150. The vaporizer body 110 may obtain the insertion event information from the NFC tag 164 and memory 191 via wireless communication circuitry 142.

The insertion event may trigger, at 406, the vaporizer body 110 to send wirelessly (e.g., via NFC) a request for additional information from the pod 150. In some implementations, the request may specify the types of requested information, such as a request for pod information, a request for information identifying the composition (e.g., mixture, recipe, etc.) of the vaporizable material in the pod (e.g., a batch ID may be mapped to a given composition), a request for filler information (e.g., vendor ID identifying the entity associated with the vaporizable material filled in the pod), a request for usage information, such as puffs taken (e.g., puff count), a time of day (e.g., a date and/or time when a puff is taken), a location where a puff is taken, an amount of energy (e.g., joules) applied to the vaporizable material, an amount (e.g., weight) of the vaporizable material in the pod, a request for pod configuration information (e.g., a target heater temperature to achieve vaporization, a ramp rate to the target temperate, resistance of the heater coil, type of heater, etc.), a request for user feedback information (e.g., experience with a vaporizable material), preferences with respect to configuration of the vaporizer and the corresponding vaporizable material, and/or other types of specific information. Alternatively, the request sent at 406 may request some, if not all, information available or stored at the pod. Alternatively, the request sent at 406 may specify a time stamp, so that the pod 150 can respond with information (which is stored at 191) having a time stamp on or after the time stamp in the request. The batch ID and the strain ID are similar in some respects in that both identify the vaporizable material. However, the batch ID identifies the composition of the vaporizable material, which may vary from batch to batch during manufacture of a given strain.

In the example of FIG. 4, the request at 406 is for all information associated with the pod (e.g., “send me all Pod info”), so the pod responds, at 408A, with pod information, such as a pod ID, pod configuration information, etc. The response, at 408B, includes vendor information, such as a vendor ID (e.g., an identifier for a vendor or filler associated with the pod), a batch ID (e.g., an identifier indicating the specific composition of the pod), product/strain information, and/or corresponding lab test results or certifications (e.g., a state lab test result, a lab test ID number for a given lab batch ID, a certificate of analysis, etc.). The response, at 408C, may include usage data, such as a puff count, a weight (or an amount) of the vaporization material in the pod (e.g., the initial amount of vaporizable material filled by the filler and/or a remaining amount after vaporization), a total energy (e.g., in joules), an age of the vaporizable material, a last used temperature (e.g., as set, configured, or selected by a user for the pod), a default temperature (e.g., set, configured, or selected by a filler, vendor, or other entity associated with the pod), and/or lock out information (e.g., an indication of whether the pod and/or vaporizer device was locked out and thus disabled for vaporization use due to a safety or other concern). The total energy may represent the total amount of joules needed to completely vaporize the vaporizable material (e.g., until the pod has dispensed the vaporizable material). Alternatively or additionally, the total energy may represent the total amount of joules expended by the heater (which represents the amount of energy applied to vaporization material providing an indication of amount remaining in the pod). The age of the vaporizable material may also be used to adjust the pod's heating configuration as well (e.g., older vaporization material, such as cannabis, may lose efficacy with age, so may require modified heating cycles to account for this change in efficacy). Although FIG. 4 depicts the responses as three separate messages 408A-C, the responses may be provided fewer or greater number of messages (e.g., a single message). In the example of FIG. 4, cartridge manufacturer (CM) data (e.g., pod ID) may be sent at 408A, filler data (e.g., batch ID) may be sent 408B, and information regarding the usage of the vaporizer sessions may be sent at 408C.

At 410, the vaporizer body 110 saves the information received at 408A-C. For example, the vaporizer body 110 may save the information received at 408A-C to memory 146 (labeled “local cache”). This information may be used to configure the vaporizer 100.

At this point, the vaporizer body 110 may be considered initialized and ready for use as the vaporizer body knows: the identity of the pod (e.g., pod ID); the specific composition of the vaporizable material in the pod (e.g., as a function of the batch ID which identifies a specific composition or mixture of a vaporizable material, such as percentage of THC, CBD, etc.); and/or how to heat the vaporizable material. As such, when a user 499 takes a draw, the vaporizer body 110 can properly heat and thus vaporize the vaporizable material.

In some implementations, when a pod is inserted at 402A-B, the pod 150 may respond by providing an initial set of information to enable a “quick start” of the vaporizer 100. This initial set may include target vaporization temperature, target “session” energy, ramp rate, and/or other basic information needed to configure vaporization. In some embodiments, the initial set may include only the target temperature. In some embodiments, the initial set may include only the target temperature and pod ID.

When the user 499 takes a puff (e.g., a drag, a draw, etc.) at 420 from the vaporizer 100, the vaporizer body 110 detects the puff using, for example, sensors 137 and 138 in the vaporizer body 110. When the puff is detected, this may trigger the vaporizer body 110 to signal, at 422, the pod 150. For example, the signal (or, e.g., message) at 422 may activate the heater circuitry 130 to initiate heating of the vaporizable material in the pod 150.

At 424, the pod 150 may store at memory 191 information related to the detected puff. This event information may include a puff count and/or other information. The puff count may be tracked to indicate usage of the pod 150. For example, a pod 150 may be filled with a vaporizable material having a certain batch ID indicative of the composition of the vaporizer material. For that vaporizer material corresponding to the batch ID, the maker of the vaporizer material (or pod filler) may determine that at a certain heater temperature, the pod 150 has only a given quantity of puffs before the pod is empty (or not usable or suitable for vaporization). Although the previous example used puff count to determine when the pod is empty, the amount of vaporizable material remaining in the pod may be determined in other ways, such as using energy consumed by the heater. The memory 191 may store other information for a user's session, such as a puff duration, a time of day, an amount of energy (e.g., joules) consumed by the heater during the puff, amount (e.g., weight) of the vaporizable material in the pod consumed during the puff, batch ID, etc.

As noted, the vaporizer body 110 may determine an amount of vaporizing material remaining in the pod 150. For example, the vaporizing body 110 may provide to a user an indication that the pod 150 is running low on vaporizable material. Similarly, the vaporizing body 110 may prevent the user from taking a puff if the pod is empty or un-usable (as attempting a vape with no or little vaporizable material may cause an unpleasant user experience).

From time to time, the vaporizer body 110 may request updated pod information from the pod 150 as shown at 426, for example. In some implementations, the request at 426 may be for only the change since the last time (e.g., since 408A-C) the pod provided information to the vaporizer body, as in the example of FIG. 4 at 426. This change information may include the current puff count, usage information, and/or other information available at the pod. However, the vaporizer 110 may request, at 426, specific types of information from the pod 150 or request some, if not all, available information from the pod.

At 428, the pod 150 responds to the vaporizer body 110 with the requested information, such as the puff count and the like. As noted, the responsive information may be carried by NFC between the NFC tag 164 and wireless communication circuitry 142.

At 430, the vaporizer body 110 may then save the received information. For example, the vaporizer body 110 may save the received information to memory 146, which is local to vaporizer body 110.

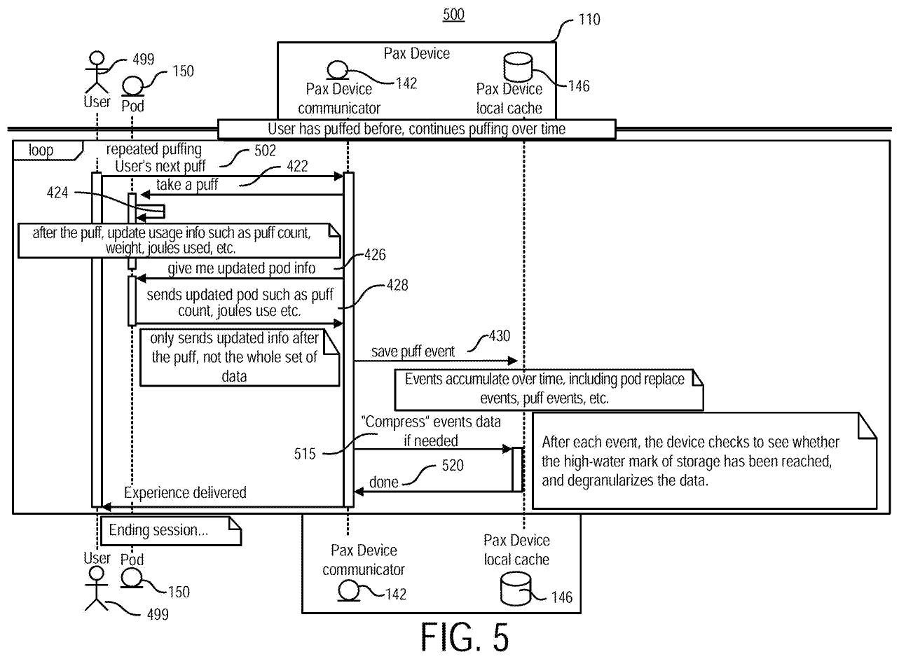

FIG. 5 depicts an example of a data exchange sequence 500 associated with the vaporizer 100. FIG. 5 is similar to FIG. 4 in some respects but includes a compression feature to free up space in memory 146, although the compression may also be applied to memory 191 as well.

At 502, a user 499 may take a plurality of drags (e.g., puffs, rips, inhales, etc.) from the vaporizer 100. After repeated use, the vaporizer body 110 may accumulate a substantial amount of information in memory 146. In some implementations, the vaporizer body 110 may apply compression, at 515, to the information in memory 146. The compression may include deleting information, although other types of data compression may be used as well.

In some implementations, the compression at 515 may include consolidating events into summary information. For example, four different events indicating four separate puffs each consuming a certain amount of energy in joules may be compressed by consolidating the four events into one summary event indicating the total number of puffs and total amount of joules consumed. Tables 1 and 2 below depict an example of this de-granularization. In the example of Table 1, memory 146 requires four memory slots, while Table 2 captures a summary of the information of Table 1 while only requiring one memory slot.

TABLE 1
4 events

| Time of Day | Energy |
|---|---|
| 12:02 AM | 1 |
| 12:04 AM | 1 |
| 12:30 AM | 1 |
| 12:50 AM | 4 |

TABLE 2
Compression

| Time of Day | Energy |
|---|---|
| 12:00-1:00 AM | 7 |

In some implementations, the compression at 515 may be triggered when the memory 146 reaches a threshold of memory used or a threshold amount of memory remaining. Alternatively or additionally, the compression may be triggered from time to time to free up space in memory. Alternatively or additionally, the compression may be triggered each time an event (e.g., information related to a puff event) is received or detected. Alternatively or additionally, the compression may be triggered by the vaporizer 100 uploading information to the user device 305 or the remote server 307. At 520, the memory 146 may acknowledge the compression by indicating that the compression has been performed. For example, the memory 146 may respond to the circuitry 142 or controller 128 with an acknowledgement.

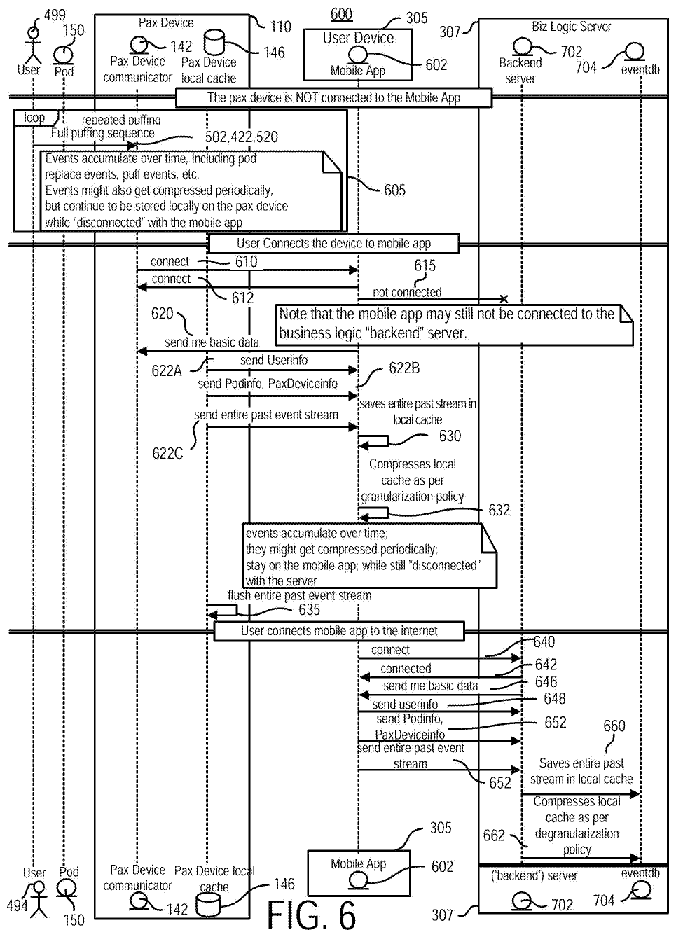

FIG. 6 depicts a data exchange sequence 600 associated with the vaporizer 100, a user device 305 including an app 602 (labeled mobile app), and a remote server 307. FIG. 6 is similar to FIG. 4 and FIG. 5 in some respects but also shows the sequence related to coupling the user device 305 and/or remote server 307.

At 605, the vaporizer 100 may not be connected to the user device 305. During this time, the vaporizer may detect one or more puffs, store information regarding the puffs, and perform compression as described above at, for example, FIG. 5 at 502 and 422-520. Although the example of FIG. 6 shows the vaporizer 100 coupling to remote server 307 via the user device 305, the vaporizer may couple to the remote server 307 without the intervening user device 305.

When the vaporizer 100 connects, at 610, to the user device 305 including the app 602, the user device 305 and the app 602 detect the connection. The connection between the user device and vaporizer may be a wired connection and/or wireless connection. For example, the user device may couple to the vaporizer via the wired connection 118, such as a universal serial bus Type C (USB-C) connection and/or the like. Alternatively or additionally, the user device 305 may couple wirelessly to the vaporizer 100. For example, the user device 305 may couple via wireless technologies, such as Bluetooth, Bluetooth Low Energy, NFC, cellular, WiFi, and/or other wireless technologies. In some implementations, the wireless technology is a short-range wireless link, such as a radio technology having low power consumption examples of which include NFC, Bluetooth Low Energy, and Bluetooth, to minimize power consumption at the vaporizer.

At 612, the user device 305 including the app 602 may respond to the connection with an acknowledgement message sent to the vaporizer 100. After this connection, the user device including the app may establish communication connections to enable the exchange of information.

At 615, the user device 305 including the app 602 may attempt to establish a connection to the remote server 307. For example, the user device 305 may attempt to couple to the remote server via wired and/or wireless links. To illustrate further, the user device 305 may be a tablet, a smart phone, or other type of processor-based device, which can couple wirelessly to the Internet and thus access the remote server 307 (which in this example, is an Internet-coupled, cloud-connected server).

In the example of FIG. 6, the connection to the remote server is not yet available at 615. This may be due to lack of a wireless or wired connection, although if the user device 305 is not registered with the remote server 307 (e.g., lacking proper authentication credentials, password, etc.), the app 602 may not be able to connect to the remote server 307. As noted, the remote server 307 may be an Internet coupled server, such as a cloud server. The remote server 307 may require authentication from a user device before access is granted. The remote server 307 may include an application server 702 and a database 704 for storing information, such as event information from the user device or vaporizer. The database 704 may also store information for a single vaporizer or a plurality of vaporizers.

At 620, the user device 305 including the app 602 sends a request to the vaporizer body 110 to send information (e.g., “send me basic data”). The request for information may be a general request for information. In some implementations, the request may be for specific types of information, such as a request for vaporizer or pod information, a request for information identifying the composition of the vaporizable material in the pod (e.g., batch ID), a request for filler information, a request for usage information (e.g., puffs taken (puff count), time of day, an amount of energy applied to the vaporizable material, an amount of the vaporizable material in the pod, etc.), a request for pod configuration, a request for user experience information, and/or other types of specific information. Alternatively, the request sent at 406 may request some, if not all, information stored memory 146.

At 622A-C, the vaporizer body 110 may respond with information, such as usage or user information, pod information (pod ID, batch ID, etc.), vaporizer device information, puff event information (e.g., puff count, etc.), and/or other information available at the vaporizer body 110. In some instances, the information may have been compressed, in which case it may represent a summary of the information as noted at Tables 1-2 above.

At 630, the user device 305 may save the information received at 622A-C locally, such as in a cache. At 632, the user device 305 may implement a compression scheme such as the compression described above with respect to 515.

In some implementations, after the vaporizer body 110 uploads information to the user device 305 as in 622A-C, the vaporizer body 110 flushes, at 635, its memory 146 by deleting some of the past events to free up memory space available. For example, the flushing may include deleting the puff events stored in memory 146 as this information has been uploaded to user device 305 from memory 146. The flushing at 635 may be in response to an indication from the user device 305 that the information provided at 622A-C has been saved successfully at the user device 305. In some implementations, the memory 191 at the cartridge may also be flushed and/or compressed. The user device 305 including the mobile application may configure one or more aspects of the vaporizer 100 based on the information provided by the vaporizer to the user device.

When the user device 305 connects, at 640, to the remote server 307, the remote server 307 may acknowledge, at 642, the connection with a message or some other indication. For example, the user device 305 may display a user interface indicating whether the remote server 307 can be accessed and, if so, whether information can be uploaded to the remote server and/or downloaded from the remote server to the user device. The information exchanged with the remote server 307 may be anonymized, so that there is no information stored at the remote server 307 that identifies the user device 305, the vaporizer 100, and/or the user of the user device or vaporizer. In some implementations, the information exchanged with the remote server 307 may be anonymized so personal identifying information of a user cannot be determined, but the anonymization may retain information regarding the identity of the vaporizers and/or cartridges. In this way, the remote server can track information for a specific vaporizer device but not retain the identity of the user of the device. In some implementations, the connection 640 represents a connection request message, in which case the remote server 307 responds with a message at 642 (which may carry information or an acknowledgment of the connection complete).

The acknowledgement received at 642 may trigger the vaporizer body 110 to push to the remote server 307 information, such as the information noted below with respect to 648-652. Alternatively or additionally, the user device 305 may wait for a request, at 646, from the remote server 307, for information.

The request for information at 646 (e.g., send me basic data) may be a general request for information or the request may specify specific types of information requested. In some implementations, the remote server 307 may send to the user device 305 (and associated mobile app) authentication or validation data to confirm an authenticated user. In some implementations, the remote server 307 may send software updates for the mobile app or other information.

At 648-652, the user device 305 may respond with the information obtained from the vaporizer 110, such as the information obtained from the vaporizer body at 622A-C.

At 660, the remote server 307 may save to cache memory or database 704 the information received by the user device 306. The remote server 307 may perform, at 662, a compression function to de-granularize the event data as noted above at 515. The information exchange depicted at FIG. 4-FIG. 6 may enable the configuration of one or more aspects of the vaporizer.

FIG. 3 and FIG. 6 demonstrate that the remote server 307 may provide a repository of vaporizer related information. This information may be stored anonymously for the users of the vaporizer to enable sharing among users and sharing with other entities, such as manufacturers, fillers, and/or the like. This information sharing may provide enhanced configuration of the vaporizers. Moreover, fillers of cartridges may be able to use the information stored at remote server 307 to facilitate automated filling of cartridges (also referred to as pods). Moreover, fillers may receive from the remote server information, such as usage data (e.g., total puffs of certain batches, temperature settings for vaporization, and user feedback, etc.) to adjust formulations in a given batch, develop new formations of the vaporizable material, provide suggested settings for the vaporizer and/or heater, and the like.

In some example embodiments, the cartridge may receive information for storage at the cartridge. For example, information may be written to memory 191 of the cartridge 150 via NFC wireless circuitry 164 at the cartridge and/or may be read from memory 191 via the NFC wireless circuitry 164 at the cartridge. For example, an NFC read and/or write device (which may also include NFC wireless circuitry) may read and/or write to the memory 191. This information may include cartridge information and/or usage information, and may be received (e.g., via a wireless transceiver) from the vaporizer body, a user device coupled to the vaporizer body, a remote server, and-or other processor-based device (e.g., a filler or manufacturer's computer). This received information may provide a configuration for the cartridge when operated with the vaporizer body. For example, a user may provide at a user device a configuration for temperature or other aspects and this may be sent to the cartridge for storage. Likewise, a remote server may provide to the vaporizer including the cartridge information which when used configured the vaporizer including the cartridge. This information may include user preference information (e.g., temperature of other information provided by the user of the vaporizer or other users, manufacturers, or fillers, of the strain contained at the cartridge).

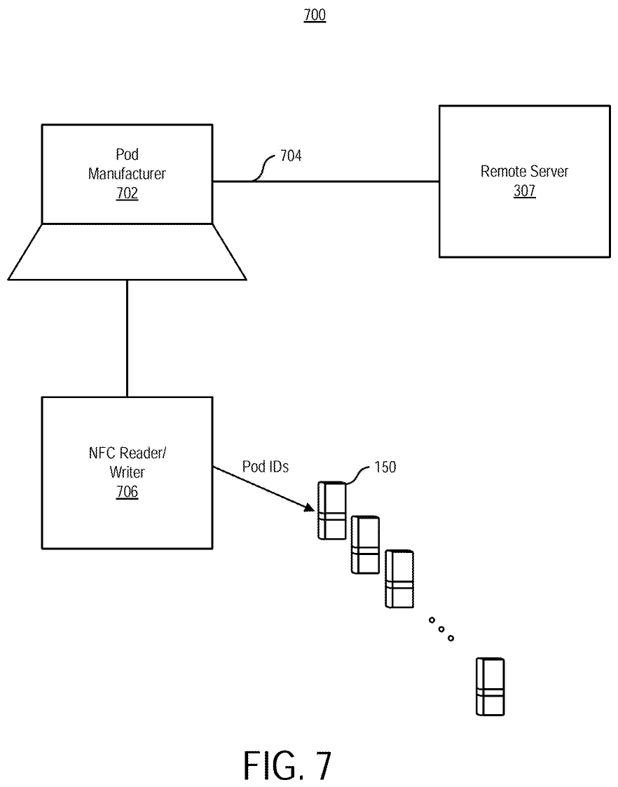

FIG. 7 depicts an example of a system 700 associated with a manufacturer of cartridges (also referred to as “pods”). The system 700 may include a processor-based device 702, such as a computer, a smartphone, a tablet, and/or the like, coupled to the remote server 307 via, for example, the Internet and/or other wired and/or wireless network. The pod manufacturer may manufacturer cartridges, such as cartridge or pod 150, and/or make the vaporizable material contained in the cartridge. Moreover, the pod manufacturer may write a pod ID to the NFC tag 164, where it is stored in memory 191. For example, an NFC reader and writer 706 may write the pod ID to the NFC tag 164. The manufacturer may manufacture a plurality of cartridges, each of which includes a unique pod ID written to the NFC tag to distinguish and identify each cartridge from other cartridges. The manufacturer may manufacture different strains of vaporizable material as well. In some example embodiments, the vaporizer body may write to the memory 119 (located in the cartridge) information disclosed herein, such as some of all of the cartridge information, some or all of the usage information, etc. For example, the cartridge may receive the information via NFC wireless (or other wireless or wired connection technology) the information, which is then written to the memory 119. The information written to memory may originate at the remote server, user device coupled to the vaporizer, and/or other devices.

In some implementations, the pod manufacturer 702 may access a web portal or other interface at the remote server 307. For example, the pod manufacturer may access the web portal by authenticating (e.g., via a login and password) and then providing to the remote server a list of the pod IDs manufactured by the manufacturer 702. Alternatively or additionally, the pod manufacturer may provide one or more strains including a strain ID identifying the specific strain and/or a batch ID identifying the composition of a specific strain (as the batch composition for a given strain may vary from batch to batch) of the vaporizable materials which can be contained in the cartridges. The remote server 307 may then save the pod IDs linked to a manufacturer ID for that specific manufacturer 702. Alternatively or additionally, the remote server 307 may save the strain and batch information to the manufacturer (or its manufacturer ID). Another pod manufacturer may similarly access the web portal at the remote server by authenticating and then provide to the remote server a list of the pod IDs and/or strains and batch information, so that the remote server 307 may save the pod IDs and/or strain and batch information for the other manufacturer as well. In this way, the remote server may track the manufacture of pods and, in particular, the composition of the vaporizable material. Moreover, the remote server may push the strain information to other devices in the system. For example, the remote server may push the strain and/or batch information to an automated pod filling machine to facilitate pod filling or push the strain and/or batch information to a user device, such as a smart phone, couple to the vaporizer device to enable configuration of the vaporizer.

Although the previous example refers to a manufacturer, the manufacturer may be a filler of the pods as well. Moreover, although the previous example refers to the pod IDs being provided by the manufacturer, the pod IDs for each pod may be assigned by the remote server to each manufacturer. When the manufacturer 702 registers (e.g., authenticates its identity with a login and password) with the remote server 307, the remote server may assign to that specific manufacturer a pod ID, which the remote server will map to the manufacturer ID and when the pod is filled with vaporizable material the remote server will also map a strain ID and batch ID indicative of the contents of the pod to that pod ID.

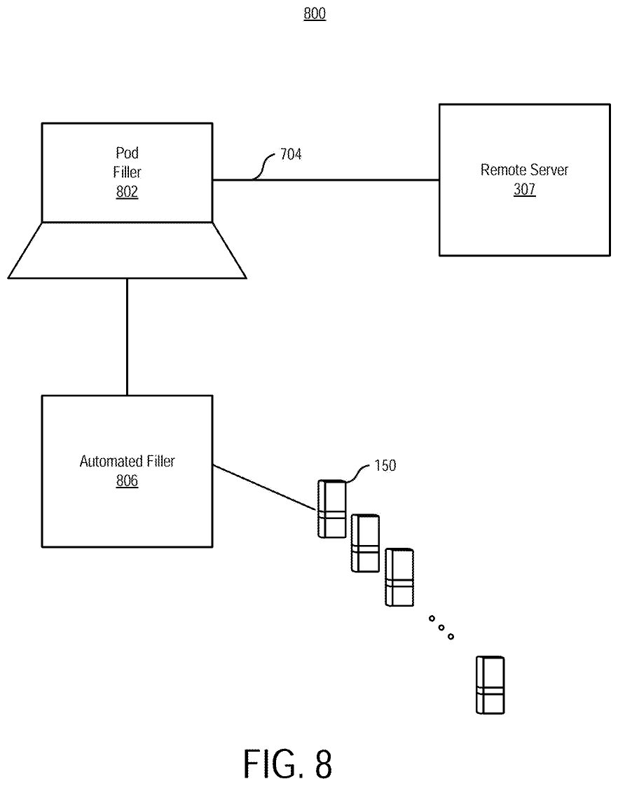

FIG. 8 depicts an example of a system 800 associated with a filler of cartridges.

In some implementations, the cartridge filler 802 includes an automated cartridge filling machine (labeled “automated filler” 806). When this is the case, the automated filler 806 may fill a plurality of pods, such as pod 150, with vaporizable material. For example, a plurality of pods may be loaded into the automated filler 806. The automated filler may include a stage that rotates each pod through stations, although other types of automated fillers may be used as well. A station may be used to fill a given pod; another station may be used to label the pod with the identity of the vaporizable material (e.g., apply a logo, trademark, etc.); and/or another station may write to the NFC tag's memory pod information, such as batch ID (or pod ID if the NFC tag 164 does not already have a pod ID), etc. The automated filler may include a processor, such as a computer, and/or the like, that couples via a network, such as the Internet, to the remote server 307.

In the example of FIG. 8, the filler's processor-based device 802 is coupled to the remote server 307 via link 704. In some implementations, the pod filler 802 may access a web portal or other interface at the remote server 307. For example, the pod filler may access the web portal by authenticating (e.g., via a login and password) and then providing to the remote server a filler ID which identifies the filler. The pod filler may also provide via the web portal at the remote server strain IDs and/or batch IDs with corresponding mixtures (e.g., recipes, compositions, or the like) of vaporizable material. For example, a filler may have a plurality of vaping material mixtures, each of which is identified with a stain ID and/or batch ID. For a given strain, there may be a strain ID for that strain, but due to batch-to-batch variations in the strain due to making the vaporizable material, a batch ID may also be used to track the batch-to-batch variations. The strain ID and/or batch ID may also be linked to other information, such as recommended target temperature for vaporization, recommended target temperature for flavor, ramp rate to target temperature, certificate of analysis, test data, etc. This information may be stored at the remote server, such as at a database at the remote server. Once a cartridge has been filled with the vaporizable material, the filler may provide, via the web portal, the pod ID mapped to the strain ID, batch ID, and/or other information as well. Thus, the remote server may have for a given pod ID detailed information, such as the manufacturer ID, strain ID, batch ID, recommended temperatures, etc.

The remote server 307 may provide to the manufacturer 702 and/or the filler 802 information as well via communication link 704 (e.g., the Internet etc.). For example, the remote server may provide vaporizer user data (which may be anonymized), such as puff counts, time of puff, feedback (from user or others) regarding likes, dislikes, feedback regarding other target temperatures or vaporizer configurations, consumption rates of the cartridges, the performance of the filler's pods in the market in comparison to other filler's pods (which may be anonymized or de-identified), etc. This information may enable the manufacturer 702 and/or the filler 802 to adjust the mixtures of existing batches or create new types of batches. Moreover, the information provided by the remote server may also allow the manufacturers and/or fillers to predict demand based on the usage patterns provided by the vaporizer devices to the remote server. As noted, the manufacturer 702 and the filler 802 may be the same entity, in which case the systems of FIG. 7 and FIG. 8 may be combined into a single system.

In some implementations, the automated filler 806 may select a batch ID from the remote server 307 (e.g., via pod filler 802 and link 704) and then obtain the corresponding mixture of the vaporizer and fill pods 150 with the retrieved recipe or mixture obtained from the remote server. For example, the remote server may provide a web portal, which allows pod filler 802 to view various batch IDs and their corresponding mixtures. The filler 802 may select a batch ID, which triggers the remote server to download the corresponding mixture for the selected batch ID to the automated filler 806, so that the selected batch is used to fill cartridges 150.

In some implementations, other pod fillers may similarly access the remote server to provide information as noted above. In some implementations, the different pod fillers batch IDs (and corresponding mixtures) are kept separate so that a filler only has access to its own batch IDs and not the other filler's batch IDs and mixtures.

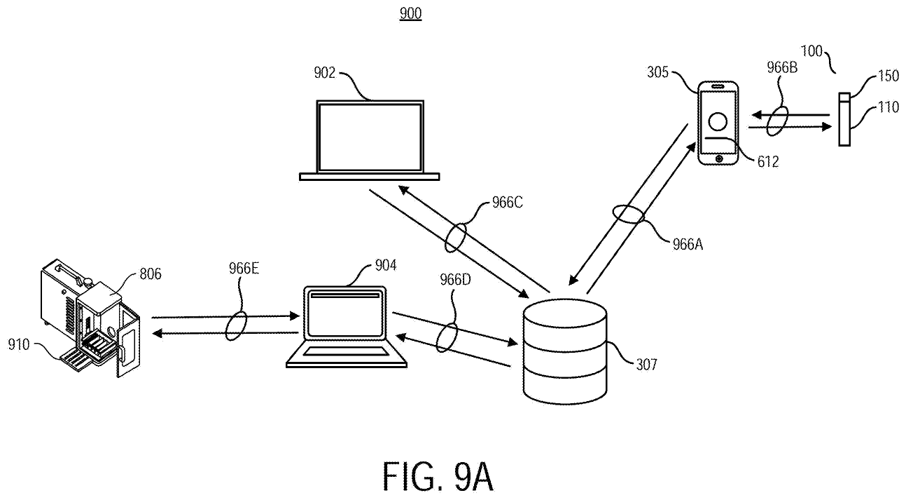

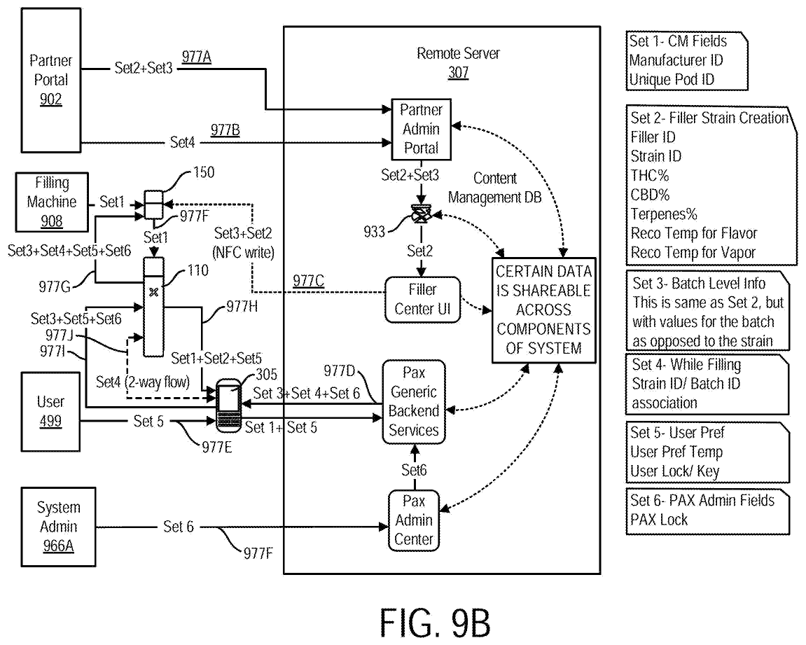

FIG. 9A depicts an example of a system 900 handling data exchanges related to aspects of the vaporizer 100.

The system 900 may include the remote server 307 coupled via wired and/or wireless links 966A to the user device 305 including the mobile application 602. The user device 305 and the application 612 may be coupled via wired and/or wireless links 966B to the vaporizer 100 (which includes the pod 150 and vaporizer body 110).

The remote server 307 may also be coupled via wired and/or wireless links to an operator application, such as partner portal 902. The partner portal 902 may provide a user interface at which information about the vaporizable material may be provided to the remote server to enable configuration of the vaporizer, filler machines, and the like. For example, a maker, such as the manufacturer 702, of vaporizable materials may enter strain information and/or batch information for the various vaporizable materials that can be contained in the pod 150. The strain and batch information along with other information may be used to configure an automated filling machine, configure aspects of the vaporizer, provide information to a user of the vaporizer, and the like.

The remote server 307 may also be coupled via wired and/or wireless links 966D to an operator application, such as production portal 904, which is further coupled via wired and/or wireless links 966E to an automated pod filling machine 806. The automated pod filing machine may fill one or more pods with vaporizable material, laser or write the pod with information (e.g., brand information, pod information, and the like), and/or write, via a wireless technology such as NFC, to the pod's memory, such as memory 191 (FIG. 2). In some implementations, the partner portal 902 and production portal 904 may both be implemented as an operator application provided by the remote server fillers, vaporizer makers, and other entities associated with the vaporizer device 100 including pod 150.

Referring again to the partner portal 902, it may be used by, for example, a maker of the vaporizable material to provide to the remote server 307 information regarding the different strains or batches of the vaporizable material. For example, the maker, such as manufacturer 702, may register with the remote server 307 (e.g., with a password and/or login ID), obtain a manufacture ID, and then provide via upload the different strains of vaporizable material to the remote server 307 to enable sharing this information among devices of system 900. To illustrate further, the maker may provide strain information to the remote server 307. This strain information may include: a filler or manufacturer identifier (ID), a strain ID, the composition of the strain (e.g., percentage of THC, percentage of CBD, percentage of Terpenes, etc.), recommended temperature (e.g., target temperature of heating of the vaporizer material) for flavor, recommended temperature for vapor, batch ID including any batch related information, and/or other information. The target temperature for flavor may be a lower temperature when compared to the target temperature for vaporization. In some implementations, there may be three temperature settings as follows: a recommended temperature (e.g., a default temperature recommended by the maker of the vaporizable material and that the device 100 is automatically set to when the pod is inserted); a recommended temperature for flavor (e.g., a lower temperature setting that will yield a better taste of the vaporizable material); and a recommended temperature for vapor (e.g., a higher temperature that will result in a larger and more condensed cloud of vapor).

To illustrate further, a maker or filler of vaporization materials may have a variety of strains, such as “Cookie Dough,” “Maui G,” and the like. And, the maker may provide information about these strains to remote server 307 to enable the information to be provided to the user devices 305, the vaporizers 100, and the production portals 904 (which may be coupled to other devices including filling machines 806, pod labelers (e.g., a printing device, laser writer, label applier, etc. for marking the strain and placing other information on the pod). In some embodiments, the filler 806 may receive one or more pod IDs from the remote server. After filling the pods, the filler may respond to the remote server with each pod ID mapped to at least the strain ID and/or batch ID of the vaporizable material filled in the cartridge.

FIG. 9B depicts a dataflow for the system 900. The partner portal 902 may provide, at 977A-B, to the remote server 307 strain creation information (labeled set 2), batch information for the strain (labeled set 3), and filling information (labeled set 4). The remote server 307 may store the information received from the partner portal at a content management system (CMS), such as a database (DB).

The remote server 307 may provide, at 977C, information, such as the pod ID, manufacturer ID, and the strain creation information (labeled sets 1 and 2) to the production portal 904 so that the automated filling machine 806 can fill the pods with the proper strains and batches, laser write information on the pods, and/or write, via NFC, information, such as set 1 and/or set 2 information, to each pod's memory. In some implementations, the automated filling machine 806 may be implemented to flash (e.g., write via NFC) information to the pod's memory and/or laser write information on the exterior of the pod (which may be filled either before or after the flashing and writing).

At 977D, the remote server 307 may provide strain and/or batch information (e.g., strain ID and/or batch ID) as well as other information (set 6) to the user device. The “Pax Lock” at set 6 refers to the ability to “lock” the vaporizer device 100 to prevent vaporization, such as the use of a pod for vaporization. The user lock and key at set 5 also represents a lock of the vaporizer so that it cannot be used for vaporization. The Pax Lock at set 6 may be a system lock triggered by an administrator of system 900 (e.g., to lock the device 100 for safety or other reasons). The set 5 lock may be a user controlled lock. For example, a user of the vaporizer device 100 may lock the vaporizer to prevent others from using the vaporizer device 100. The “Key” at set 5 represents a code used to lock and unlock the vaporizer device 110 to enable vaporization using the pod. At 977E, the user 499 may provide user preference information (set 5 which also includes the user's preferred temperature) to the user device 305, which forwards that information to the server 307. Moreover, a user, such as a system administrator 966, may provide, at 977F, to the remote server the administration information (set 6), which is also forwarded to the remote server 307.

FIG. 9B also shows the information exchanges between the pod 150, device body 110, and user device 305. At 977F, the pod 150 may provide, via NFC, to the vaporizer body 110 set 1 information, such as pod ID and/or manufacturer ID. At 977G, the vaporizer body 110 may provide, via NFC, information such as set 3, 4, 5, and 6 information. At 977H, the vaporizer body 110 may also forward to the user device 305 set 1, 2, and 5 information, and the user device 305 may forward, at 977I-J, to the vaporizer device body 110 set 3-6 information.

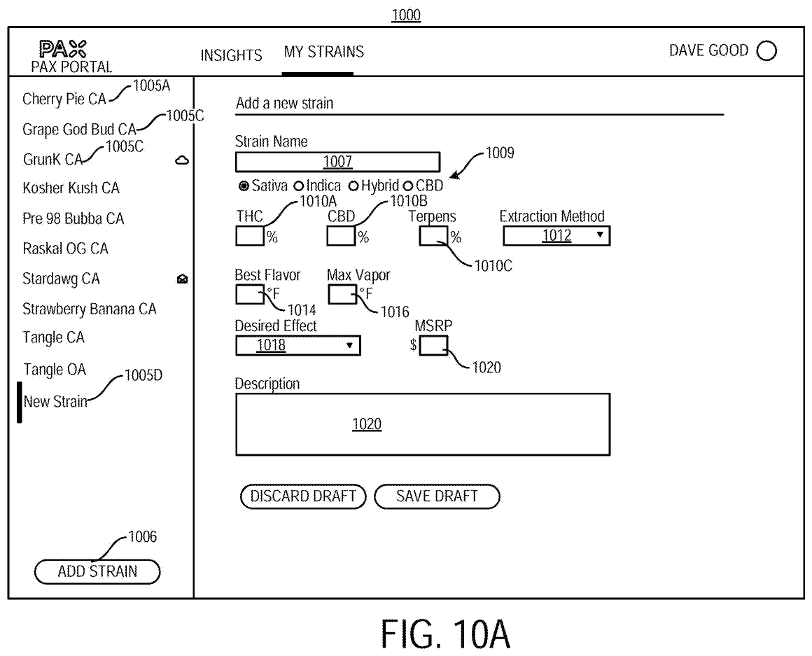

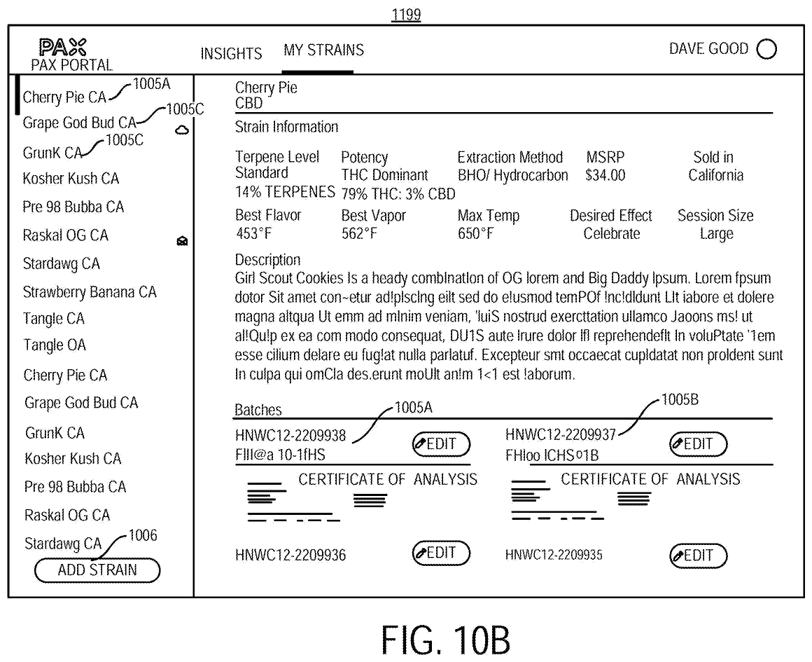

FIG. 10A depicts an example of a user interface 1000 presented at partner portal 902. The user interface 100 allows strains of vaporable material, such as strain 1005D, to be registered in the system 900 including remote server 307. In the example of FIG. 10A, the partner portal also shows other strains 1005A-C that this partner portal 902 has previously provided to the remote server 307. In operation, different makers of vaporizable material have corresponding partner portals through which those makers can privately load and manage their strains. At the user interface 1000, the following strain information may be entered: strain name 1007, strain type 1009, composition 1010A-D, extraction method 1012, recommended temperature for best flavor 1014, recommended temperature for best vapor 1016, desired effect 1018 after consuming vaporizable material (e.g., calm, alert, drowsy, etc.), price 1020, and other information, which may be related to the strain. To add the strain and push it to the remote server 307, the add strain 1006 is selected, which triggers the strain to be provided and registered with the remote server for the given partner at portal 902.

FIG. 10B depicts another example of a user interface 1999 presented at a given partner portal 902. This example shows that for a given strain there can be various batches 1055A-B. For example, each time a batch of strain “Cherry Pie” is made, the composition may vary, so the partner portal may be used to enter or edit the batch specific information which is tracked by the remote server according to the batch ID. Thus, for any given strain, such as Cherry Pie, there will be a manufacture ID (which identifies the partner making the vaporizable material), a plurality of strain IDs (which identify the manufacturers strains), and, for each strain ID, and/or one or more batch IDs (which identify batches for a strain as each batch may vary slight with respect to recommended temperatures, composition, etc.).

Referring again to FIG. 9A, the production portal 904 may be used by a filler of pods to enable automated filling of pods with a filling machine 806. The filler of the pods may, as noted, be the same or different entity from the manufacturer at the partner portal 902.

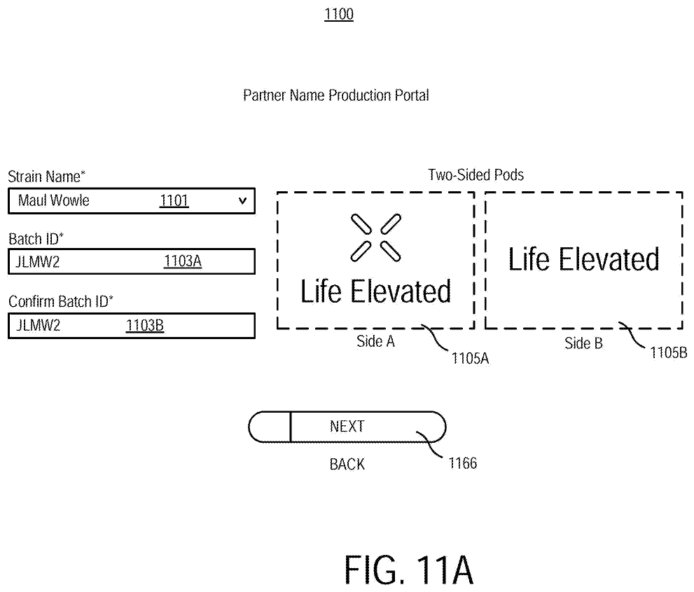

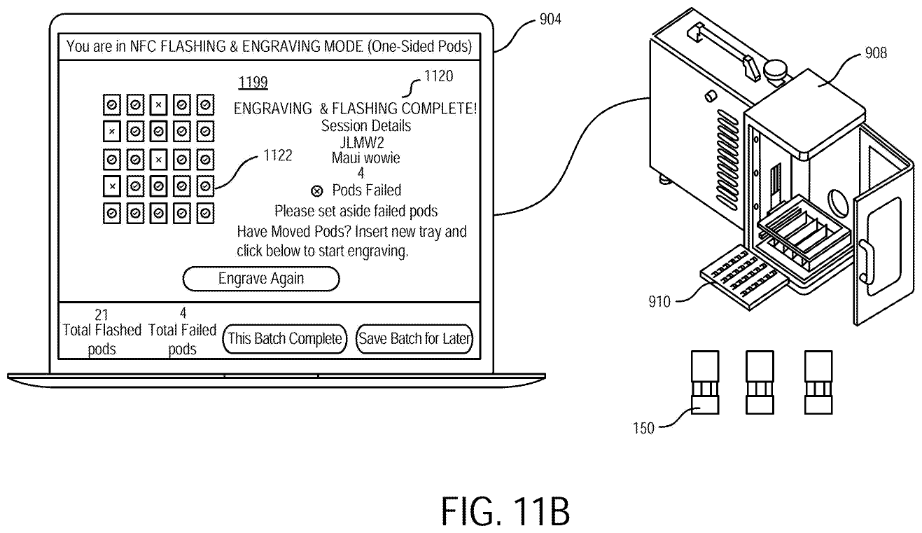

In the example of FIG. 9A, a tray of pods 910 are inserted into the filling machine 806. As noted above, the filling machine may be configured to perform one or more of the following: fill pods, write to the pod's NFC memory, laser write to the exterior of the pods, and/or perform other operations on the pods. The pods are empty of vaporizable material, so they are ready to be filled. FIG. 11A depicts a user interface 1100 presented at the production portal 904. For a given manufacturer and/or filler associated with the production portal 904, the user interface 1100 includes one or more strain names which can be selected at 1101 for filling, and for the selected strain 1101 one or more corresponding batch IDs. The user interface 1100 may also allow graphics 1105A-B to be printed on the pod itself (in this example, two sides of the pod are printed with the selected graphics). The production portal 904 may obtain, for a given manufacturer ID and/or filler ID, the strain names, batch IDs, graphics, and other information from the remote server.

When next is selected 1166 at FIG. 11A, it may trigger the filling machine 806 to begin filling the pods in tray 910 with the selected vaporizer material mapped to the strain ID 1101 and batch ID 1103A-B. The filling machine may also automatically write the graphics 1105A-B on the sides of the pods as part of the filling process. Moreover, the filling machine may write (or flash) via NFC information into each pod's memory as part of the filling process. As noted above, the filling machine may have multiple stages for filling, laser writing graphics, performing NFC writes, etc. During the NFC write stage, the information written into the pod may include a pod ID (which may be unique to each pod), a manufacturer ID (which identifies the maker of the vaporizable material at partner portal 902), the strain information: such as a filler identifier (ID), a strain ID, the composition of the strain, recommended temperate for flavor, recommended temperature for vapor, batch related information, and/or other information. The pod ID may be unique, such that each pod can be tracked. The unique pod ID may be unique to a region, such as a country, a state, or other region, so that the pod ID identifies the pod from other pods in the region, although the unique pod ID may be unique globally as well. In some embodiments, the pod ID is assigned and provided by the remote server 307 to the production portal 904.

FIG. 11B depicts another user interface 1198 presented on the production portal 904. The user interface 1198 shows the status of the pod filling at tray 910. In this example, the user interface 1199 provides an indication 1120 of whether the engraving of the graphics and writing (or flashing) of information into the pod's NFC is complete. The user interface 1198 also provide an indication of whether a pod was successfully filled 1122. In the example of FIG. 11B, an “X” indicates a failure of some sort in the pod filling and a “✓” indicates the pod filling is successful. The user interface 1198 presents pod filling success or failure indications in a format that mirrors the tray 910 so that the failed pod can be readily identified in the tray 910.

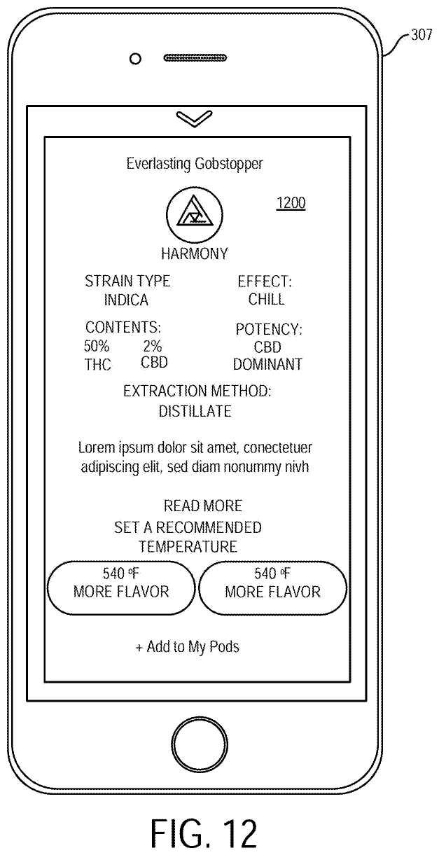

Referring again to FIG. 9A, the remote server 307 may exchange information with the user device 305, application 602, and/or vaporizer 100. For example, the information provided via the partner portal 902 for a given strain may be provided to the user device 305 for presentation to the user of the vaporizer 100 as shown in FIG. 12. FIG. 12 depicts an example of a user interface 1200 presented on user device 307. In this example of FIG. 12, the information depicted was initially provided by the partner portal (see, e.g., FIG. 10A), stored at the remote server for that given partner or manufacturer, and then provided to the user device 307 for presentation. The partner portal may also allow link other media, such as instructional videos, music, promotional material, etc. to enable the maker to control the brand and provide for an enhanced user experience, and this additional media may be presented at 1200 as well.

The user device 305 and/or vaporizer 100 may also provide to the remote server 307 user preference information, such as a user's preferred temperature for a given strain and batch, etc., and user usage information, such as puff count, etc.

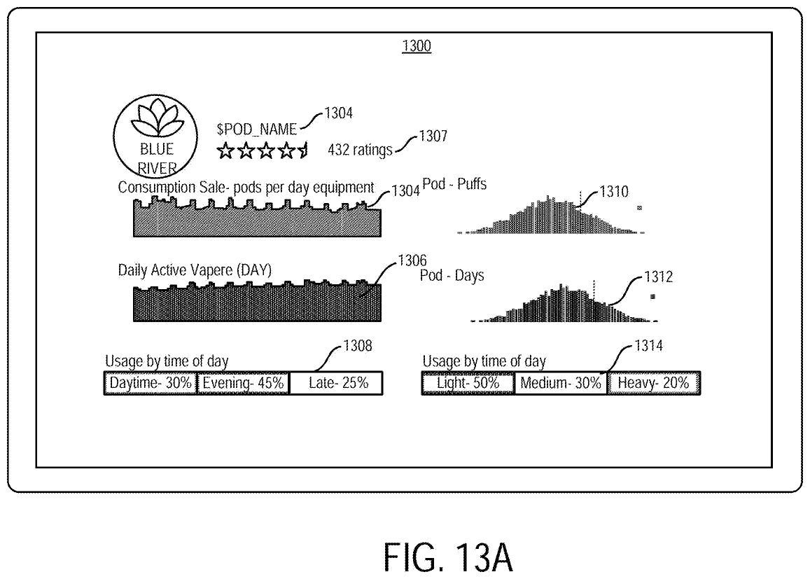

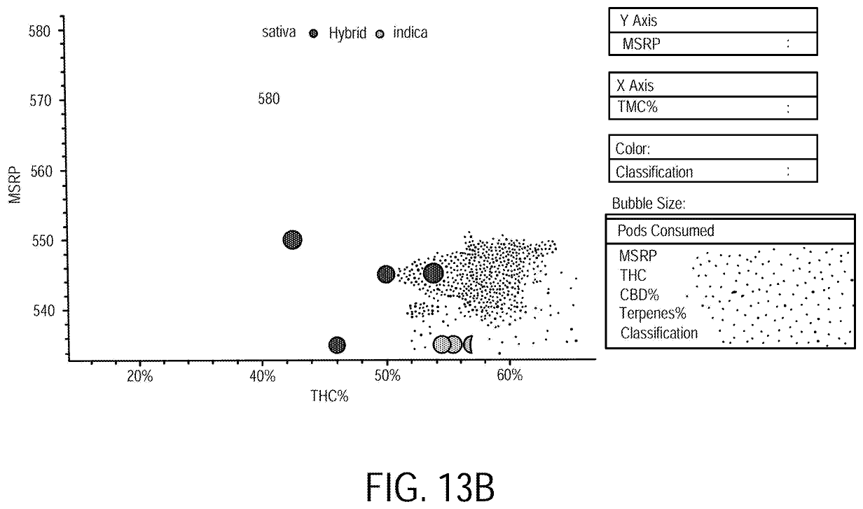

The remote server 307 may also analyze data stored at the remote server. For example, each strain and/or batch for a given manufacture may be analyzed. FIG. 13A depicts an example of a user interface 1300 providing analytics, such as a strain name 1304, a user rating 1307 for the strain, a consumption rate of a pod 1304, a quantity of active users of the vaporizers per day 1306, a time of day usage of the pod 1308, a lifetime of the pod in terms of puffs 1310 or days 1312, and the pod's usage by type of consumer (e.g., heavy vaporizer user, light vaporizer user, etc.). The user interface 1300 may be presented at partner portal 902, although it may be presented at other devices as well. FIG. 13B depicts another example user interface providing analytics on pod consumption including price and vaporization material content (e.g., percentages of THC, CBD, etc.).

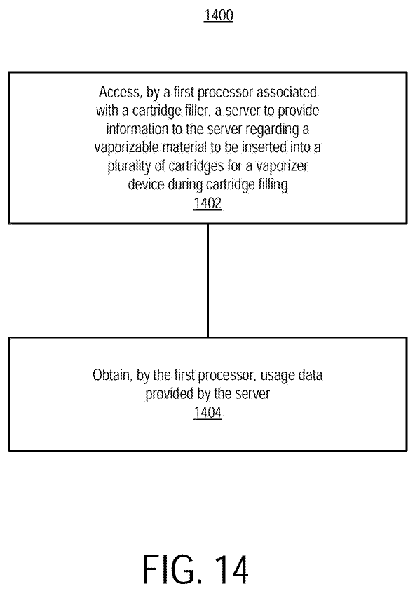

FIG. 14 depicts an example of a process associated with a cartridge filler. The description of FIG. 14 also refers to FIG. 8.

At 1402, a processor, such as a processor 802, may access a server, such as remote server 307. This access may allow the processor 802 to provide to the server 307 information regarding a vaporizable material to be inserted into a plurality of cartridges for corresponding vaporizer devices during cartridge filling. For example, a filler may provide information such as pod identifier (ID) identifying a cartridge containing the vaporizable material, a batch identifier indicating a composition of the vaporizable material, a mass of an empty cartridge, a vendor identifier, a product identifier, a strain code, a mass of a filled cartridge, a viscosity, a default temperature setting, a minimum temperature setting, a maximum temperature setting, a tetrahydrocannabinol (THC) content percentage, a cannabidiol (CBD) percentage, a terpene percentage, a fill date, and/or other information.

At 1404, the processor 802 may obtain information, such as usage data and/or other types of information, from the server 307. For example, the processor 802 associated with the filler may obtain usage data. Examples of usage data includes a total quantity of the puffs taken for a cartridge, a total puff time for a cartridge, a total energy delivered to a cartridge, a time and date information for puffs on a cartridge, and/or user feedback information for a cartridge, and/or other information. The remote server may thus be used to provide a repository of information for the cartridges that can be used with the vaporizer. This information may be used to configure the vaporizer for use. The filler may use the information to enable tracking usage, modifying formulations for different strains (or strengths) of vaporizable material inserted into cartridges, generating suggested vaporizer configurations (e.g., heating rate, vaporization temperature, session duration, etc.) for a given vaporizable material being used in a cartridge. For example, usage data may be provided to an automated filler (or a processor of the automated filler) to configure the automated filler. To illustrate further, the usage data may provide information to modify the strain, mixture, etc. used in the vaporizable material being used to fill the cartridges. Likewise, feedback from the usage data may be used to revise the target heating temperature for a given vaporizable material, and this revised target temperature may be stored in memory at the corresponding cartridge being filled with the given vaporizable material.

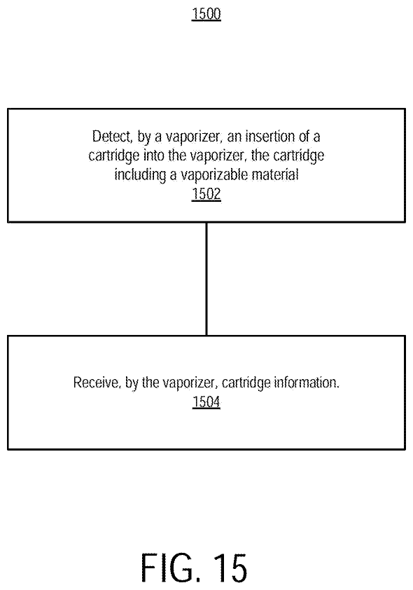

FIG. 15 depicts an example of a process associated with a vaporizer. The description of FIG. 15 also refers to FIG. 2-FIG. 5.

At 1502, the vaporizer 110 may detect an insertion of a cartridge 150 (which includes a vaporizable material) into the vaporizer. As noted at 402A-B and 404 for example, the vaporizer may detect the insertion of the cartridge. The insertion of the cartridge may take a variety of forms, such as screwing the cartridge into the vaporizer body, inserting the cartridge having a friction fit or magnetic coupler to the vaporizer body, and/or other mechanisms of coupling the cartridge to the vaporizer body.

At 1504, the vaporizer 110 may receive cartridge information provided by the cartridge 150. For example, the cartridge 150 may provide via a wireless link, such as NFC, Bluetooth Low Energy, or other type of link (e.g., wired) cartridge information. The cartridge information may include a pod identifier identifying the cartridge containing the vaporizable material, a batch identifier indicating a composition of the vaporizable material, a vendor identifier, product information, a lab test result, a lab certification, usage data of the cartridge, age of the vaporizable material, a last used temperature, a mass of an empty cartridge, a vendor identifier, a product identifier, a strain code, a mass of a filled cartridge, a viscosity of the vaporizable material contained in the cartridge, a default temperature setting, a minimum temperature setting, a maximum temperature setting, a tetrahydrocannabinol (THC) content percentage, a cannabidiol (CBD) percentage, a terpene percentage, a fill date, and/or lock out information. As noted, the cartridge information may enable the vaporizer to configure one or more aspects of the vaporization of the vaporizable material by the vaporizer.

In some example embodiments, a vaporizer device may include a cartridge. The cartridge may include a cartridge body defining, at least in part, a reservoir configured to contain vaporizable material, a vaporizing assembly positioned within the cartridge body in fluid communication with the reservoir, the vaporizing assembly configured to vaporize the vaporizable material, and a mouthpiece coupled to a proximal end region of the cartridge body. The vaporizer device may also include a vaporizer body. The vaporizer body may include a receptacle configured to receive at least a portion of the cartridge, control circuitry comprising at least one processor and at least one memory disposed within the vaporizer body. The at least one memory may include instructions which when executed cause operations including storing an event indicative of detection of an insertion of the cartridge into the vaporizer body; requesting, from the cartridge, cartridge information; receiving, in response to the request, the cartridge information stored at the cartridge; and configuring, based at least in part of the received cartridge information, the vaporizer device for vaporizing of the vaporizable material.

The cartridge information may be received via a first near field communication circuitry at the cartridge and a second near field communication circuitry at the vaporizer body. The cartridge information may include a pod identifier identifying the cartridge containing the vaporizable material and/or a batch identifier indicating a composition of the vaporizable material. The at least one memory may include instructions which when executed cause operations including requesting, from the cartridge and after a detection of a puff of the vaporizer device, usage information of the vaporizer device and storing, at the at least one memory, the usage information received from the cartridge. The at least one memory may include instructions which when executed cause operations including compressing, by the vaporizer device, the cartridge information and/or the usage information after a detection of a puff, a detection of a cartridge insertion, and/or a threshold of available storage is reached. The at least one memory may include instructions which when executed cause operations including establishing one or more connections with a user device and/or a remote server and sending, via the one or more connections, the cartridge information including the usage information to the user device and/or the remote server. The usage information may include a total quantity of the puffs taken for the cartridge, a total puff time for the cartridge, a total energy delivered to the cartridge, a time and date information for puffs on the cartridge, and/or user feedback information for the cartridge.

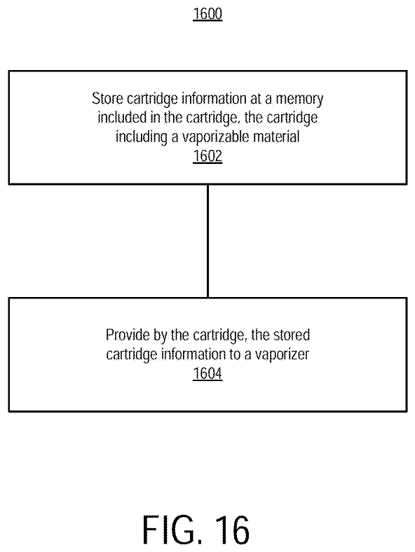

FIG. 16 depicts an example of a process associated with a cartridge. The description of FIG. 16 also refers to FIG. 2-FIG. 5.

At 1602, the cartridge 150 may store cartridge information at a memory, such as memory 191, included in the cartridge. The cartridge may include a vaporizable material. At 1604, the cartridge 150 may provide the stored cartridge information to a vaporizer 110. The cartridge may provide the cartridge information after the insertion of the cartridge into the vaporizer as noted above at 402A-408C. The cartridge information may be provided at other times as well, such as in response to a request (e.g., request 406 and 426) or after a puff event (e.g., after 424). As noted, the cartridge information may enable the vaporizer to configure one or more aspects of the vaporization of the vaporizable material by the vaporizer.

In some example embodiments, the cartridge includes a cartridge body defining, at least in part, a reservoir configured to contain vaporizable material; a vaporizing assembly positioned within the cartridge body in fluid communication with the reservoir, the vaporizing assembly including a heating element configured to heat and cause vaporization of the vaporizable material into air drawn into a vaporizer device along an air flow path; a mouthpiece coupled to a proximal end region of the cartridge body; circuitry comprising a controller, at least one memory storing cartridge information, and a wireless transceiver, the circuitry causing operations comprising sending, in response to a request from a vaporizer body, cartridge information to the vaporizer body; and/or sending, in response to a puff, usage information to the vaporizer body.

The sending may be triggered by insertion of the cartridge into the vaporizer body. The at least one memory is memory, and the wireless transceiver comprises near field communication circuitry disposed at an end of the cartridge opposite to the mouthpiece. The cartridge information and/or usage information may be sent via is provided via a wireless transceiver. The cartridge information may include a pod identifier identifying a cartridge containing the vaporizable material and/or a batch identifier indicating a composition of the vaporizable material. The usage information may include a total quantity of the puffs taken for the cartridge, a total puff time for the cartridge, a total energy delivered to the cartridge, a time and date information for puffs on the cartridge, and/or user feedback information for the cartridge. The usage information may be sent to the vaporizer body as an updated after at least one puff. The cartridge information may be sent to the vaporizer body in response to insertion of the cartridge into the vaporizer body or after at least one puff of the vaporizer device. The cartridge information may be sent to the vaporizer body in response to a request received from the vaporizer device. The stored cartridge information and/or usage information at the cartridge may be compressed.

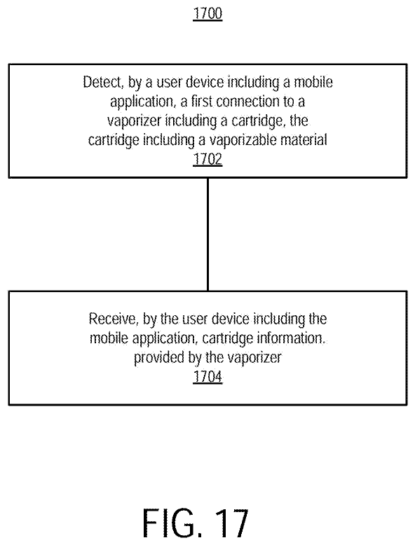

FIG. 17 depicts an example of a process associated with a user device, such as a smartphone, tablet, or other processor-based device. The description of FIG. 17 also refers to FIG. 2-FIG. 6.

At 1702, the user device 305 including the mobile application 602 may detect a first connection to a vaporizer 110 including a cartridge 150 (which includes a vaporizable material). As noted above at 610-612, a first connection between the user device 305 and vaporizer 110 may be established and, e.g., detected.

At 1704, the user device 305 including the mobile application 602 may receive cartridge information provided by the vaporizer 110 over the first connection. As noted above with respect to 622A-C for example, the cartridge information may be received by the user device 305. The first connection may be a wired and/or a wireless connection, such as Bluetooth technology (which may enable low power use with the vaporizer). The cartridge information is received in response to the first connection being established and/or in response to a request received from the user device including the mobile application. In some implementations, the request indicates to the cartridge to provide a certain type of cartridge information, updated cartridge information, or all cartridge information available at the vaporizer device including the cartridge. In some implementations, the user device provides the cartridge information to a remote server, such as remote server 307. The cartridge information may be provided to the remote server in response to a second connection establishment to the remote server and/or in response to a request received from remote server. As noted, the cartridge information provided to the user device may enable the user device to configure one or more aspects of the vaporization of the vaporizable material by the vaporizer (e.g., heating rate, vaporization temperature, session duration, etc.).

In some example embodiments, the user device may be configured to establish a first connection to a vaporizer device including a cartridge, the cartridge including a vaporizable material; receive, in response to a first request sent to the vaporizer device, a first set of cartridge information stored at the vaporizer device, the first set of cartridge information including a pod identifier uniquely identifying the cartridge, a manufacturer identifier identifying a manufacturer of the vaporizable material included in the cartridge, and a strain information indicating a strain of the vaporizable material included in the cartridge; send, in response to a second connection being established to a remote server, the first set of cartridge information to the remote server; receive, from the remote server and in response to the sending of the first set of cartridge information to the remote server, a second set of cartridge information including batch information indicative of a composition of the vaporizable material in the cartridge; and/or configure, based on the first set of cartridge information and/or the second set of cartridge information, the vaporizer device including the cartridge to vaporize the vaporizable material.

The first connection may be via a Bluetooth wireless connection. The strain information may include a filler identifier indicating an identity of a filler of the cartridge, a strain identifier indicating a strain of the vaporizable material in the cartridge, a percentage of tetrahydrocannabinol contained in the vaporizable material, a percentage of cannabidiol contained in the vaporizable material, a percentage of terpenes contained in the vaporizable material, a recommended temperature for flavor when heating the vaporizable material, and a recommended temperature for vapor when heating the vaporizable material. The first set of cartridge information may include usage information, wherein the usage information includes a total quantity of the puffs taken for a cartridge, a total puff time for a cartridge, a total energy delivered to a cartridge, a time and date information for puffs on a cartridge, and/or user feedback information for a cartridge. The first request may indicate to the vaporizer device to provide a certain type of cartridge information, provide updated cartridge information, or provide all cartridge information available at the vaporizer device including the cartridge. The first set of cartridge information may be sent in response to a second request received from the remote server. The second request may indicate to the user device including the mobile application to provide to the remote server a certain type of cartridge information, updated cartridge information, or all cartridge information available at the user device including the mobile application. A lock may be received from the remote server and in response to the sending of the first set of cartridge information to the remote server. The lock may be an indication to lock operation of the user device and/or the vaporizer device to prevent vaporization of the vaporizable material.

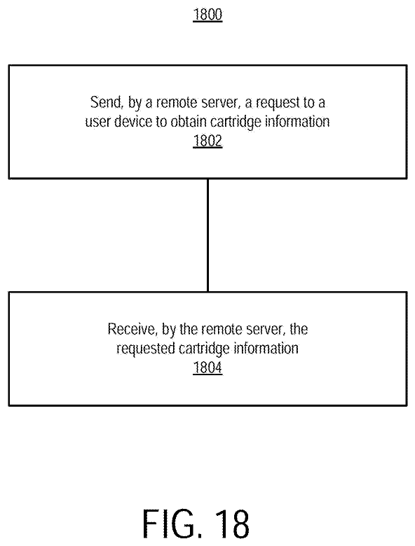

FIG. 18 depicts an example of a process associated with a user device, such as a smartphone, tablet, or other processor-based device. The description of FIG. 18 also refers to FIG. 6-FIG. 7.

At 1802, the remote server may send a request to a user device to obtain cartridge information. For example, the remote server 307 may send a request to user device 305 to provide cartridge information. The request may indicate to the user device to provide a certain type of cartridge information, updated cartridge information, or all cartridge information available at the user device. In response, the remote server 307 may receive the requested cartridge information. The cartridge information may receive by the server 307 in response to a wired and/or a wireless connection being established between the remote server and the user device including the mobile application. In some implementations, the remote server may enable access to cartridge information stored at the remote server and providing cartridge information and/or usage data to other processor-based devices.

In some example embodiments, the remote server may receive, from a first user device, information indicating a strain of a vaporizable material included in a cartridge configured to contain the vaporizable material; send, to the first user device, a pod identifier to enable a filler of the cartridge to associate the pod identifier to the cartridge and the strain of the vaporizable material included in the cartridge; and receive, from the first user device and after the cartridge is filled with the vaporizable material, a strain identifier mapped to the pod identifier, the strain identifier indicating the strain of the vaporizable material included in the filled cartridge; and/or store the pod identifier mapped to the strain identifier to enable configuration of a vaporizer device.

The received information may further include batch information indicating a composition of the vaporizable material included in the cartridge. The batch information may include a filler identifier indicating an identity of a filler of the cartridge, a strain identifier indicating a strain of the vaporizable material in the cartridge, a percentage of tetrahydrocannabinol contained in the vaporizable material, a percentage of cannabidiol contained in the vaporizable material, a percentage of terpenes contained in the vaporizable material, a recommended temperature for flavor when heating the vaporizable material, and a recommended temperature for vapor when heating the vaporizable material. The first user device may be coupled to a filling machine configured to fill the cartridge with the vaporizable material, store the pod identifier in a memory of the cartridge, and store a strain identifier indicative of the strain of the vaporizable material in the memory of the cartridge. The first user device may be coupled to an near field communication writer to store to a memory at the cartridge a manufacturer identifier, the pod identifier, a filler identifier, a strain identifier, a percentage of tetrahydrocannabinol contained in the vaporizable material, a percentage of cannabidiol contained in the vaporizable material, a percentage of terpenes contained in the vaporizable material, a recommended temperature for flavor when heating the vaporizable material, and/or a recommended temperature for vapor when heating the vaporizable material. Usage data associated with the vaporizer device may be received by the remote server. The usage data may include a total quantity of the puffs taken for the cartridge, a total puff time for a cartridge, a total energy delivered to the cartridge, a time and date information for puffs on the cartridge, a user preference associated with a configuration of the vaporizer device, and/or user feedback information for the cartridge. The remote server may provide, to a user device coupled to the vaporizer device, the usage data, the pod identifier, and/or the strain identifier to enable the user device to configure the vaporizer device for vaporization of the vaporizable material in the cartridge. The remote server may send, to the user device coupled to the vaporizer device, a lock indication to lock operation of the user device and/or the vaporizer device to prevent vaporization of the vaporizable material.

Referring again to FIG. 3 depicting the vaporizer device 100, the user device 305, and the remote server 307, the user of the vaporizer device may want information regarding the provenance of the vaporizable material in the cartridge. To that end, there may be provided certificate of analysis (COA) information which may include test information for the vaporizable material. For example, the remote server 305 may include for a vaporizable material strain and a corresponding batch of that strain the COA information. If there are a plurality of batches for the vaporizable material strain, the COA information for each of the corresponding batches may be stored at the remoter server 305.

To illustrate further by way of an example, a pod manufacture, such as pod manufacturer 702 as shown at FIG. 7, may have each batch of a strain tested to verify the ingredients of the batch of vaporizable material. The testing may include, for example, determining the percentage of THC, CBD, terpenes, and/or other ingredients of the vaporizable material. Moreover, the testing may also test for unwanted, undesired, unexpected, or harmful ingredients. For example, the testing may test for toxins, pesticides, metals, or other substances. In any case, the pod manufacturer 702 may provide the COA information (which includes the test information) to the remote server 307.

In some example embodiments, the COA information may be stored in the remote server 307.

In some example embodiments, the remote server 307 may provide to a user device 305 at least a portion of the stored COA information as test information.

In some example embodiments, the remote server 307 may provide to the user device 305 the test information in response to a request from the user device 305. In some example embodiments, the request includes information identifying the strain and/or the batch of the vaporizable material contained in the cartridge 150.

In some example embodiments, if the vaporizable material contained in the cartridge 150 does not have COA information stored at the remote server 307, the remote server may send to the user device 305 (and/or the vaporizer device 100) a message. This message may provide a warning that the provenance of the vaporizable material cannot be confirmed. Alternatively or additionally, the message may inhibit use of the vaporizable material. For example, the remote server may send a message to the user device to inhibit (e.g., shut down, inhibit heating, etc.) the vaporizer device 100 from vaporizing the vaporizable material.

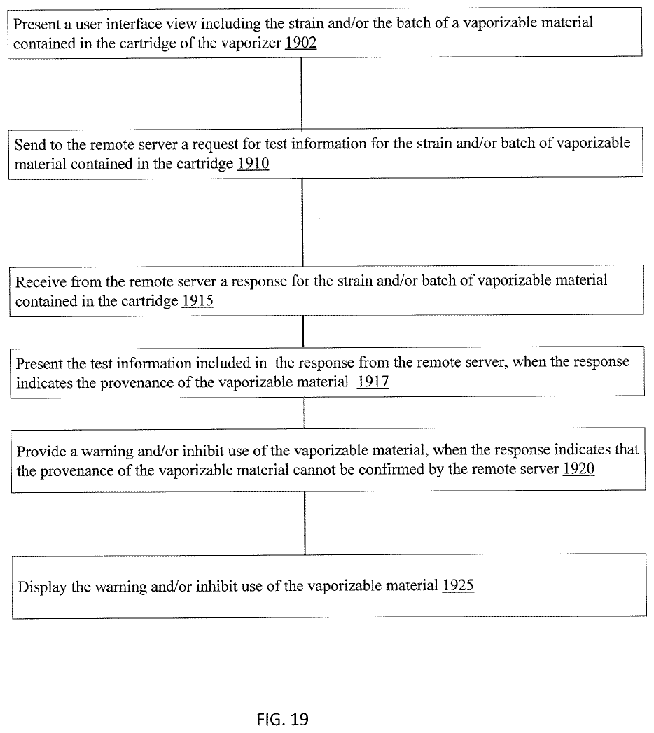

FIG. 19 depicts an example of a process configured at the user device 305 for handling the testing information for the vaporizable material contained in a cartridge 150 that is inserted into the vaporizable body 110, in accordance with some example embodiments. The description of FIG. 19 also refers to FIGS. 3, 7, and 20A-C.

At 1902, the user device 305 may present a user interface view including the strain and/or the batch of a vaporizable material contained in the cartridge 150 of the vaporizer 100. For example, when cartridge 150 is inserted into the vaporizer 100, the cartridge may provide, via wireless circuitry 142, vaporizable material strain and/or batch information to the vaporizer body and/or user device. Alternatively or additionally, the cartridge may provide to the vaporizer body and/or user device a pod identifier, which can be forwarded to the remote server 307. When this is the case, the remote server responds to the user device (and/or vaporizer body) with the strain and/or batch information for presentation at the user interface view. Alternatively or additionally, the user device may be used to search for strain and/or batch information at the remote server, in which case the remote server responds to the user device with search results including the strain and/or batch information for presentation at the user interface view. If the strain and/or batch of the vaporizable material contained in the cartridge is listed in the search results, the strain or batch may be selected as the vaporizable material contained in the cartridge.

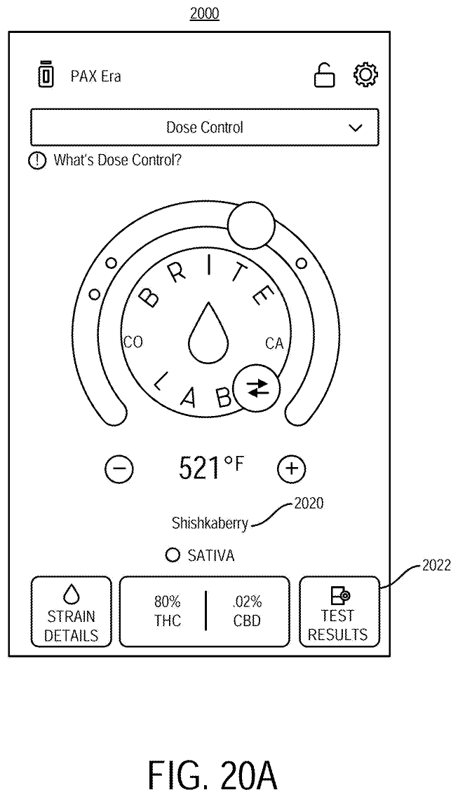

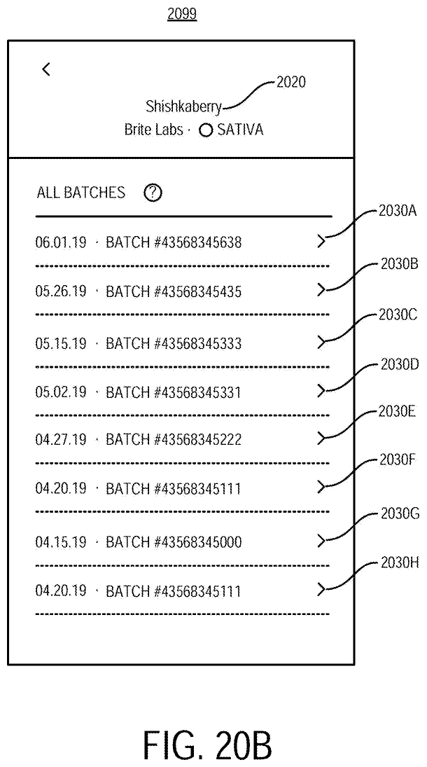

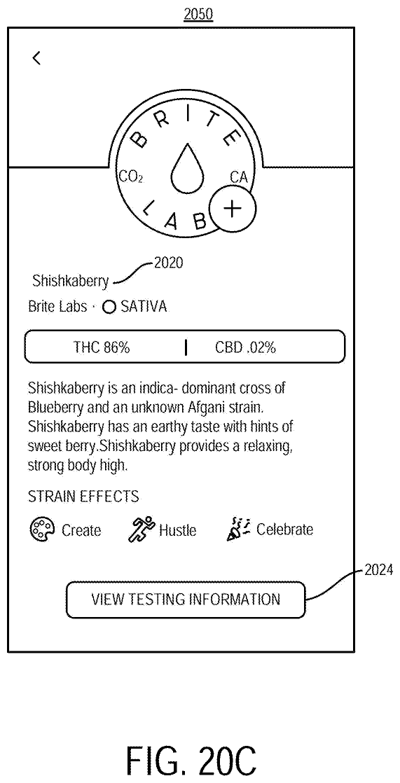

FIG. 20A depicts an example of a user interface view 2000 including strain information 2020 for the vaporizable material contained in cartridge 150. The user interface view may comprise a page, such as an HTML page, generated for presentation on a display.

For example, when cartridge 150 is inserted into the vaporizer 100, the cartridge may provide, via wireless circuitry 142, vaporizable material strain and/or batch information to the vaporizer body and/or user device. Or, the remote server may as noted respond with the strain and/or batch information for presentation at the user interface view. As shown at FIG. 20A, the strain and/or batch information may be presented at a user interface view at the user device. In this example, the strain Shishkaberry shown at FIG. 20A. FIG. 20B depicts another example of a user interface view 2099 including batch information 2030A-H for the vaporizable material strain 2020 which may be contained in cartridge 150. FIG. 20B may represent information provided by the remote server to the user device in response to a request for a search as noted above. FIG. 20C depicts another example of user interface view 2000 including strain information 2020 for the vaporizable material contained in cartridge 150.

At 1910, the user device 305 may send to the remote server 307 a request for test information for the strain and/or batch of vaporizable material contained in the cartridge 150. For example, a selection of a user interface element 2022 (FIG. 20A) may trigger the user device to send to the remote server 307 the request for test information for the strain and/or batch of vaporizable material in cartridge 150 (which in this example is Shishkaberry 2020). To illustrate further, a selection of a user interface element 2030A (FIG. 20B) may trigger the user device to send to the remote server 307 the request for test information for the strain and/or batch of vaporizable material in cartridge 150. Likewise, a selection of a user interface element 2024 (FIG. 20C) may trigger the user device to send to the remote server 307 the request for test information for the strain and/or batch of vaporizable material in cartridge 150.

In some embodiments, the user interface elements 2022 and 2024 may not be presented or may not be enabled when the remote server 307 does not include the COA information for the strain and/or batch of vaporizable material in cartridge 150. Likewise, if the remote server 307 does not include the COA information for a strain's batch of vaporizable material, the user interface element for that batch may be disabled or may not be presented at FIG. 20B. For example, if the strain 2020 does not have COA information stored at the remote server for batch #43568345638, the user interface element 2030A may not be presented or may not be enable (e.g., disabled, grayed out, not selectable, etc.), but if the strain 2020 does have COA information stored at the remote server for batch #43568345635, the user interface element 2030B would be presented and able to be selected.

In some embodiments, the request sent at 1910 includes the pod ID of the vaporizable material in cartridge 150. Alternatively or additionally, the request sent at 1910 includes the strain ID of the vaporizable material in cartridge. Alternatively or additionally, the request sent at 1910 includes the batch ID of the vaporizable material in cartridge.

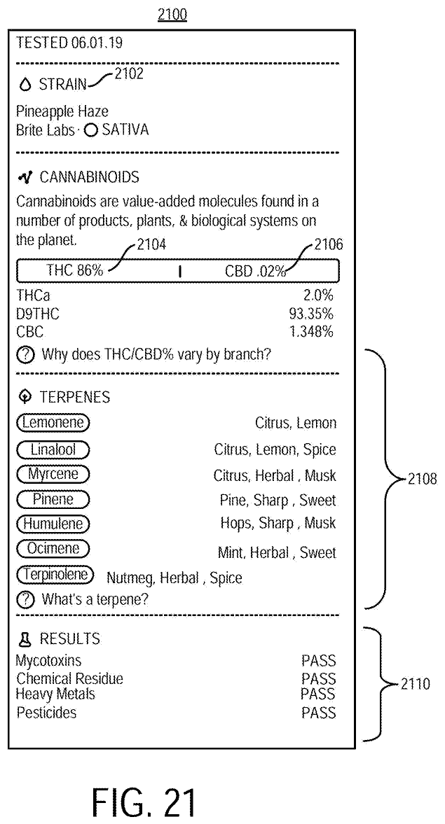

At 1915, the user device 305 may receive from the remote server 307 a response for the strain and/or batch of vaporizable material contained in the cartridge 150. For example, the remote server may receive the request sent at 1910 and then search for stored strain and/or batch information. If found, the remote server may respond with an indication that the strain and/or batch contained in the cartridge does have provenance information in the form of the stored COA. If the remote server does include the stored COA, the response to the user device may further include at least a portion of the COA information. An example of a portion of the COA information is presented as test information at FIG. 21.

FIG. 21 depicts an example of a user interface view 2100 including a portion of the COA information presented as test information. For example, the user interface view 2100 may present, for a strain 2102 or batch (which may be contained in the cartridge 150) test information. This test information may list the vaporizable materials ingredients including the corresponding concentration, such as the THC percentage 2104, CBD percentage 2106, and/or other ingredient in the vaporizable material. In the example of FIG. 21, the test information includes a terpenes profile 2108. Moreover, the test information may include one or more other test results 2010. In the example of FIG. 21, the test results 2010 includes mycotoxins, chemical residues, heavy metals, and pesticide tests. In some implementations, the test results 2010 includes mycotoxins, chemical residues, heavy metals, pesticides, microbials, microbiologicals, pesticide residue, residual solvents, myotoxins, filth and foreign materials, foreign matter and/or materials, and/or foreign matter and seeds tests, and/or the like. Although the other test results are depicted as pass or fail tests, the actual test results may be presented as well. Moreover, the content of the test information at FIG. 21 is only an example as other information may be presented to indicate the provenance, safety, and/or the like of the vaporizable material contained in cartridge 150.

If the remote server does include the stored COA, the user device 305 may receive from the remote server 307 a response indicating that the provenance of the vaporizable material contained in the cartridge 150 cannot be confirmed. As indicated, if the remote server 307 does not have COA information for a strain or a given batch of the strain, the remote server cannot confirm the provenance of the stain and corresponding batch.

When the remote server includes the COA information for the strain and/or batch, the user device 305 may present the test information included in the response from the remote server 307. For example, the user device may present the test information as shown at FIG. 21, so that the provenance of the vaporizable material may be viewed.

At 1920, the user device 305 may provide a warning that the provenance of the vaporizable material cannot be confirmed and/or inhibit use of the vaporizable material, response indicating that the provenance of the vaporizable material contained in the cartridge 150 cannot be confirmed. For example, if the response does not include test information, the user device may present, at 1920, on a display of the user device a message such as a warning that the provenance of the vaporizable material contained in the cartridge 150 cannot be confirmed. Alternatively or additionally, if the response does not include test information, the vaporizer may provide an indication, such as haptic feedback or visual feedback via LEDs. Alternatively or additionally, the user device may inhibit, at 1925, use of the cartridge 150 by inhibit heating at the vaporizer device 100 until a cartridge having a verifiable provenance can be confirmed by the remote server.

To illustrate further while referring to FIG. 3, the user device 305 may include a display, at least one processor, and at least one memory including program code which when executed by the at least one processor causes the user device to establish a first connection to a vaporizer device 100 including a cartridge 150 (which includes a vaporizable material). The user device may send to remote server 307 a request for test information for the vaporizable material. This request may be in the form of an explicit request for test information, such as a request for a COA for a given stain and batch or for a given pod. In response to the request, the user device may receive the test information indicating a provenance of the vaporizable material. FIG. 21 depicts an example of this test information presented at a display of the user device, although the test information may be presented in other formats and include other types of information. However, if the remote server does not include COA information for the vaporizable material in the cartridge, the response from the remote server may indicate that there is not test information or COA for that vaporizable in the cartridge. For example, if the remote server does not include COA information for the vaporizable material in the cartridge, the response from the remote server may include a warning or other indication for the user device. As noted, the response that there is no COA for the vaporizable material may prompt a warning to be presented on the user device display, inhibiting use of the vaporizer device 100, provide a visual or haptic alert (e.g., LEDs flashing in a predetermine pattern or a haptic pre-determined pattern), or other actions at the vaporizer device to alert the user.

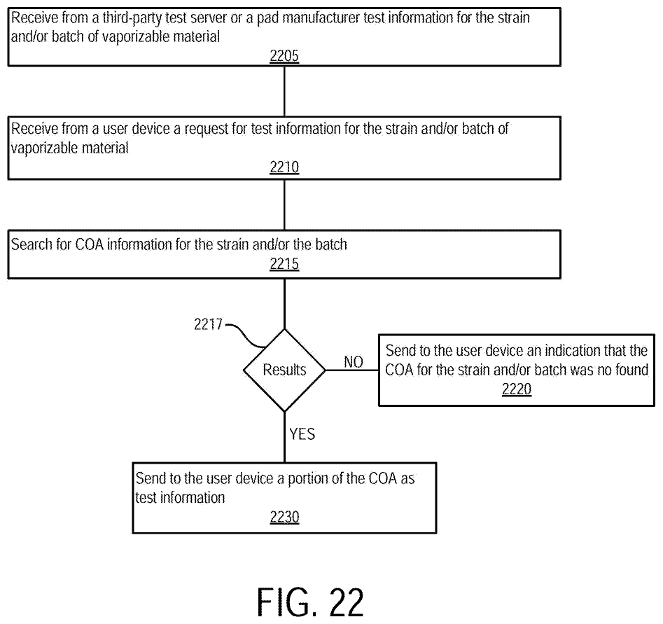

FIG. 22 depicts an example of a process configured at the remote server 307 for checking the provenance of a strain and/or batch of the vaporizable material in a cartridge 150 inserted into the vaporizable body 110, in accordance with some example embodiments.

At 2205, the remote server 307 may receive for a strain and/or batch test information for the vaporizable material. For example, a pod manufacturer may provide the COA information for each strain and, of there are a plurality of batches for the stain, COA information for the batch(es). The testing performed for the COA may be performed by the pod manufacturer 702, although one or more third parties, such as an independent laboratory, may perform the testing shown at FIG. 21. The test information may be stored at a database at (or coupled to) the remote server.

At 2210, the remote server 307 may receive, from the user device 305, the request for test information for the strain and/or batch as described above with respect to 1910.

When the request is received, the remote server 307 may search, at 2215, for the COA for the strain and/or batch associated with the request. For example, the request may include the stain ID and/or batch ID, which can be used to search the database for the COA for the strain and/or batch. If the request includes the pod ID, the remote server 307 may search its database of pod IDs to identify the strain and/or batch of the vaporizable material in cartridge 150.

If the remote server 307 obtains search results includes the strain and/or batch for the vaporizable material in cartridge (Yes at 2217), the remote sever generates a response and then sends the response including at least a portion of the COA, such as the test information depicted at FIG. 21. If the remote server 307 cannot identify the strain and/or batch for the vaporizable material in cartridge (No at 2217), the remote sever generates a response and then sends, at 2220, the response including an indication that strain and/or batch could not be found. As such, the provenance of the vaporizable material cannot be confirmed.

In some examples, the vaporizable material may include a viscous liquid such as, for example a cannabis oil, CBD oil, hemp oil, or other types of vaporizable materials. In some variations, the cannabis oil comprises between 40-100% cannabis oil extract, although the percentage of cannabis oil may be higher and/or lower as well. The viscous oil may include a carrier for improving vapor formation, such as propylene glycol, glycerol, etc., at between 0.01% and 25% (e.g., between 0. 1% and 22%, between 1% and 20%, between 1% and 15%, and/or the like). In some variations the vapor-forming carrier is 1,3-Propanediol. A cannabis oil may include a cannabinoid or cannabinoids (natural and/or synthetic), and/or a terpene or terpenes derived from organic materials such as for example fruits and flowers. For example, any of the vaporizable materials described herein may include one or more (e.g., a mixture of) cannabinoid including one or more of: CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), Tetrahydrocannabinol, Cannabidiol (CBD), Cannabinol (CBN), one or more Endocannabinoids (e.g., anandamide, 2-Arachidonoylglycerol, 2-Arachidonyl glyceryl ether, N-Arachidonoyl dopamine, Virodhamine, Lysophosphatidylinositol), and/or a synthetic cannabinoids such as one or more of: JWH-018, JWH-073, CP-55940, Dimethylheptylpyran, HU-210, HU-331, SR144528, WIN 55,212-2, JWH-133, Levonantradol (Nantrodolum), and AM-2201. The oil vaporization material may include one or more terpene, such as Hemiterpenes, Monoterpenes (e.g., geraniol, terpineol, limonene, myrcene, linalool, pinene, Iridoids), Sesquiterpenes (e.g., humulene, farnesenes, farnesol), Diterpenes (e.g., cafestol, kahweol, cembrene and taxadiene), Sesterterpenes, (e.g., geranylfarnesol), Triterpenes (e.g., squalene), Sesquarterpenes (e.g, ferrugicadiol and tetraprenylcurcumene), Tetraterpenes (lycopene, gamma-carotene, alpha- and beta-carotenes), Polyterpenes, and Norisoprenoids. For example, an oil vaporization material as described herein may include between 20-80% cannabinoids (e.g., 30-90%, 40-80%, 50-75%, 60-80%, etc.), 0-40% terpenes (e.g., 1-30%, 10-30%, 10-20%, etc.), and 0-25% carrier (e.g., polyethylene glycol).

In any of the oil vaporization materials described herein (including in particular, the cannabinoid-based vaporization materials), the viscosity may be within a predetermined range. The range may be between about 30 cP (centipoise) and 115 KcP (kilocentipoise), between 30 cP and 200 KcP, although higher viscosities and/or lower viscosities may be implemented as well. For example, the viscosity may be between 40 cP and 113 KcP. Outside of this range, the vaporizable material may fail in some instances to wick appropriately to form a vapor as described herein. In particular, it is typically desired that the oil may be made sufficiently thin to both permit wicking at a rate that is useful with the apparatuses described herein, while also limiting leaking (e.g., viscosities below that of ˜40 cP might result in problems with leaking).

Although the disclosure, including the figures, described herein may described and/or exemplify these different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

1-110. (canceled)

111. A vaporizer device comprising:
a cartridge comprising:
a cartridge body defining, at least in part, a reservoir configured to contain vaporizable material,
a vaporizing assembly positioned within the cartridge body in fluid communication with the reservoir, the vaporizing assembly configured to vaporize the vaporizable material, and
a mouthpiece coupled to a proximal end region of the cartridge body; and
a vaporizer body comprising:
a receptacle configured to receive at least a portion of the cartridge, and
control circuitry comprising at least one processor and at least one memory disposed within the vaporizer body, the at least one memory including instructions which when executed cause operations comprising:
storing an event indicative of detection of an insertion of the cartridge into the vaporizer body;
requesting, from the cartridge, cartridge information;
receiving, in response to the request, the cartridge information stored at the cartridge; and
configuring, based at least in part on the received cartridge information, the vaporizer device for vaporizing of the vaporizable material.

112. The vaporizer device of claim 111, wherein the cartridge information is received via a first near field communication circuitry at the cartridge and a second near field communication circuitry at the vaporizer body.

113. The vaporizer device of claim 111, wherein the cartridge information comprises a pod identifier identifying the cartridge containing the vaporizable material and/or a batch identifier indicating a composition of the vaporizable material.

114. The vaporizer device of claim 111, wherein the operations further comprise:
requesting, from the cartridge and after a detection of a puff of the vaporizer device, usage information of the vaporizer device.

115. The vaporizer device of claim 111, wherein the operations further comprise:
storing, at the at least one memory, the usage information received from the cartridge.

116. The vaporizer device of claim 111, wherein the operations further comprise:
compressing, by the vaporizer device, the cartridge information and/or the usage information after a detection of a puff, a detection of a cartridge insertion, and/or a threshold of available storage is reached.

117. The vaporizer device of claim 111, wherein the operations further comprise:
establishing one or more connections with a user device and/or a remote server; and
sending, via the one or more connections, the cartridge information and/or the usage information to the user device and/or the remote server.

118. The vaporizer device of claim 117, wherein the usage information includes at least one of the following: a total quantity of puffs taken from the cartridge, a total puff time for the cartridge, a total energy delivered to the cartridge, a time and date information for the puffs taken from the cartridge, and/or user feedback information regarding the vaporizable material.

119. The vaporizer device of claim 111, wherein the receptacle is configured to mate and electrically connect with the cartridge.

120. The vaporizer device of claim 111, wherein the cartridge information is stored in a memory comprised in a near field communication circuitry at the cartridge.

121. A cartridge for a vaporizer, the cartridge comprising:
a cartridge body defining, at least in part, a reservoir configured to contain vaporizable material;
a vaporizing assembly positioned within the cartridge body in fluid communication with the reservoir, the vaporizing assembly including a heating element configured to heat and cause vaporization of the vaporizable material into air drawn into a vaporizer device along an air flow path;
a mouthpiece coupled to a proximal end region of the cartridge body; and
circuitry comprising a controller, at least one memory storing cartridge information, and a wireless transceiver, the circuitry causing operations comprising:
sending cartridge information to the vaporizer body; and
sending usage information to the vaporizer body.

122. The cartridge of claim 121, wherein the wireless transceiver comprises near field communication circuitry disposed at an end of the cartridge opposite to the mouthpiece, and wherein the at least one memory is memory included in the near field communication circuitry.

123. The cartridge of claim 121, wherein the cartridge information and/or usage information is sent via the wireless transceiver.

124. The cartridge of claim 121, wherein the cartridge information comprises a pod identifier identifying a cartridge containing the vaporizable material and/or a batch identifier indicating a composition of the vaporizable material.

125. The cartridge of claim 121, wherein the usage information includes a total quantity of puffs taken from the cartridge, a total puff time for the cartridge, a total energy delivered to the cartridge, a time and date information for the puffs taken from the cartridge, and/or user feedback information regarding the vaporizable material.

126. The cartridge of claim 121, wherein the usage information is sent to the vaporizer body after at least one puff of the mouthpiece and/or after a request is received from the vaporizer body.

127. The cartridge of claim 121, wherein the cartridge information is sent to the vaporizer body in response to insertion of the cartridge into the vaporizer body, after at least one puff of the mouthpiece, and/or after a request is received from the vaporizer body.

128. The cartridge of claim 121, wherein the operations further comprise:
receiving, at the cartridge, the cartridge information and/or usage information, wherein the cartridge information is received from the vaporizer body, a user device coupled to the vaporizer body, and/or a remote server.

129. The cartridge of claim 128, wherein the cartridge information and/or usage information is received via the wireless transceiver.

130. An apparatus comprising:
at least one processor and at least one memory including program code which when executed by the at least one processor causes operations comprising:
establishing a first connection to a vaporizer device including a cartridge, the cartridge including a vaporizable material;
receiving, in response to a first request sent to the vaporizer device, a first set of cartridge information stored at the vaporizer device, the first set of cartridge information including a pod identifier uniquely identifying the cartridge, a manufacturer identifier identifying a manufacturer of the vaporizable material included in the cartridge, and strain information indicating a strain of the vaporizable material included in the cartridge;
sending, in response to a second connection being established to a remote server, the first set of cartridge information to the remote server;
receiving, from the remote server and in response to the sending of the first set of cartridge information to the remote server, a second set of cartridge information including batch information indicative of a composition of the vaporizable material in the cartridge; and
configuring, based on the first set of cartridge information and/or the second set of cartridge information, the vaporizer device including the cartridge to vaporize the vaporizable material.
